(19) United States
(12) Patent Application Publication
(10) Pub. No.: US 2016/0235752 A1
(43) Pub. Date: Aug. 18, 2016

(54) COMPOSITIONS, METHODS OF USE, AND METHODS OF TREATMENT

(71) Applicants: THE UNIVERSITY OF NEW ORLEANS, New Orleans, LA (US); BOARD OF SUPERVISORS OF LOUISIANA STATE UNIVERSITY AND AGRICULTURAL AND MECHANICAL COLLEGE, Baton Rouge, LA (US)

(72) Inventors: BRANKO S. JURSIC, NEW ORLEANS, LA (US); DONNA M. NEUMANN, NEW ORLEANS, LA (US)

(73) Assignees: University of New Orleans, New Orleans, LA (US); Board of Supervisors of Louisiana State University And Agricultural and Mechanical College, Baton Rouge, LA (US)

(21) Appl. No.: 15/025,680

(22) PCT Filed: Sep. 29, 2014
(86) PCT No.: PCT/US14/58031

§ 371 Date: Mar. 29, 2016

Related U.S. Application Data
(60) Provisional application No. 61/884,237, filed on Sep. 30, 2013.

(51) Int. Cl. A61K 31/515

(52) U.S. Cl. CPC A61K 31/515

(57) ABSTRACT

Embodiments of the present disclosure provide for compositions including an antimicrobial agent, pharmaceutical compositions including the antimicrobial agent, methods of treatment of an infection, methods of treatment using compositions or pharmaceutical compositions, and the like.

### CLAIM OF PRIORITY TO RELATED APPLICATION

This application claims priority to co-pending U.S. provisional application entitled “Novel Hydrazones of Carbonyl-Pyrimidinetriones as Potent Antimicrobial Drugs” having Ser. No. 61/884,237, filed on Sep. 30, 2013, which is entirely incorporated herein by reference.

### BACKGROUND

The mortality rates for invasive fungal infections are 20-40% for Candida albicans, 50-90% for Aspergillus fumigatus, and 20-70% for Cryptococcus neoformans. The availability of effective antifungal drug is crucial because most patients with fungal infections are immune compromised and their immune system is not efficient in the clearance of the infection. Unfortunately the number of potent antifungals is limited. Thus there is a need for new antifungals (antimicrobial agents).

### SUMMARY

Embodiments of the present disclosure provide for compositions including an antimicrobial agent, pharmaceutical compositions including the antimicrobial agent, methods of treatment of an infection, methods of treatment using compositions or pharmaceutical compositions, and the like.

In an embodiment, a composition, among others, includes: an antimicrobial agent having the following structure:

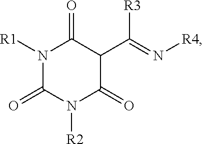

wherein R1 is selected from the group consisting of: H, alkyl, aryl, (CH₂)ₙCOOH with n=1-6, OH, and O-alkyl, where each of alkyl and aryl groups is independently optionally substituted or unsubstituted; wherein R2 is selected from the group consisting of: H, alkyl, aryl, (CH₂)ₙCOOH with n=1-6, OH, and O-alkyl, where each of alkyl and aryl groups is independently optionally substituted or unsubstituted; wherein R3 is selected from the group consisting of: H, alkyl, aryl, and COOH, where each of alkyl and aryl groups is independently optionally substituted or unsubstituted; wherein R4 is selected from the group consisting of:

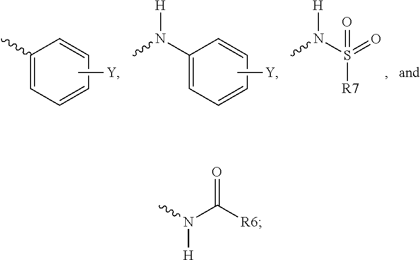

wherein Y is selected from the group consisting of: H, OH, NO₂, COOH, halogen, alkyl, and O-alkly, where each alkyl is independently optionally substituted or unsubstituted, wherein R6 is selected from the group consisting of: alkyl, O-alkyl, O-aryl, and aryl, where each of alkyl and aryl groups is independently optionally substituted or unsubstituted; and wherein R7 is selected from the group consisting of: alkyl, O-alkyl, O-aryl, and aryl, where each of alkyl and aryl groups is independently optionally substituted or unsubstituted.

In an embodiment, a pharmaceutical composition, among others, includes: a therapeutically effective amount of an antimicrobial agent, or a pharmaceutically acceptable salt of the antimicrobial agent, and a pharmaceutically acceptable carrier, to treat an infection, wherein the antimicrobial agent has the following structure:

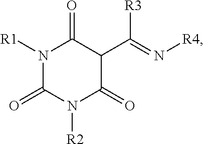

wherein R1 is selected from the group consisting of: H, alkyl, aryl, (CH₂)ₙCOOH with n=1-6, OH, and O-alkyl, where each of alkyl and aryl groups is independently optionally substituted or unsubstituted; wherein R2 is selected from the group consisting of: H, alkyl, aryl, (CH₂)ₙCOOH with n=1-6, OH, and O-alkyl, where each of alkyl and aryl groups is independently optionally substituted or unsubstituted; wherein R3 is selected from the group consisting of: H, alkyl, aryl, and COOH, where each of alkyl and aryl groups is independently optionally substituted or unsubstituted; wherein R4 is selected from the group consisting of:

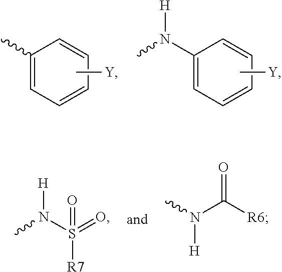

wherein Y is selected from the group consisting of: H, OH, NO₂, COOH, halogen, alkyl, and O-alkly, where each alkyl group is independently optionally substituted or unsubstituted, wherein R6 is selected from the group consisting of: alkyl, O-alkyl, O-aryl, and aryl, where each of alkyl and aryl groups is independently optionally substituted or unsubstituted; and wherein R7 is selected from the group consisting of: alkyl, O-alkyl, O-aryl, and aryl, where each of alkyl and aryl groups is independently optionally substituted or unsubstituted.

In an embodiment, a method of treating an infection, among others, includes: delivering to a subject in need thereof, a pharmaceutical composition, wherein the pharmaceutical composition includes a therapeutically effective amount of an antimicrobial agent, or a pharmaceutically acceptable salt of the antimicrobial agent, and a pharmaceutically acceptable carrier, to treat the infection, wherein the antimicrobial agent has the following structure:

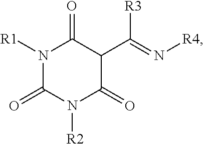

wherein R1 is selected from the group consisting of: H, alkyl, aryl, (CH₂)ₙCOOH with n=1-6, OH, and O-alkyl, where each of alkyl and aryl groups is independently optionally substituted or unsubstituted; wherein R2 is selected from the group consisting of: H, alkyl, aryl, (CH₂)ₙCOOH with n=1-6, OH, and O-alkyl, where each of alkyl and aryl groups is independently optionally substituted or unsubstituted; wherein R3 is selected from the group consisting of: H, alkyl, aryl, and COOH, where each of alkyl and aryl groups is independently optionally substituted or unsubstituted; wherein R4 is selected from the group consisting of:

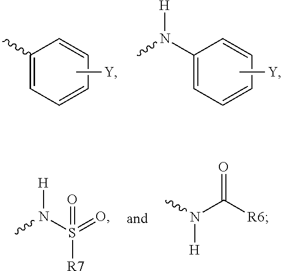

wherein Y is selected from the group consisting of: H, OH, NO₂, COOH, halogen, alkyl, and O-alkly, where each alkyl group is independently optionally substituted or unsubstituted, wherein R6 is selected from the group consisting of: alkyl, O-alkyl, O-aryl, and aryl, where each of alkyl and aryl group is independently optionally substituted or unsubstituted; and wherein R7 is selected from the group consisting of: alkyl, O-alkyl, O-aryl, and aryl, where each of alkyl and aryl group is independently optionally substituted or unsubstituted.

Other compositions, methods, features, and advantages will be, or become, apparent to one with skill in the art upon examination of the following drawings and detailed description. It is intended that all such additional structures, systems, methods, features, and advantages be included within this description, be within the scope of the present disclosure, and be protected by the accompanying claims.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure.

FIGS. 1A and 1B illustrates cytotoxicity assays of selected compounds.

FIGS. 2A and 2B illustrates BA22 activity is enhanced when Candida growth depends upon non-fermentable carbon sources. C. albicans strain SC5314 (FIG. 2A) or C. glabrata strain CS117.93 (FIG. 2B) were grown in medium with 2% glucose (YPD), 3% glycerol (YPG) or 3% ethanol (YPE) at pH 6.5, in dose response experiments with BA22. After 24 hours incubation at 30° C., growth was measured by OD₆₀₀ₙₘ. Growth at each concentration of BA22 is shown as % growth relative to the minus drug (DMSO) control. Data shown is representative of two repeat experiments.

### DISCUSSION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of organic chemistry, biochemistry, microbiology, molecular biology, pharmacology, medicine, and the like, which are within the skill of the art. Such techniques are explained fully in the literature.

Prior to describing the various embodiments, the following definitions are provided and should be used unless otherwise indicated.

### DEFINITIONS

Unless otherwise defined, all technical and scientific terms used herein have the same meaning as commonly understood by one of ordinary skill in the art of microbiology, molecular biology, medicinal chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

The term “substituted” refers to any one or more hydrogens on the designated atom that can be replaced with a selection from the indicated group, provided that the designated atom's normal valence is not exceeded, and that the substitution results in a stable compound. The term “substituted,” as in “substituted alkyl”, “substituted aryl,” “substituted heteroaryl”, and the like means, unless defined otherwise herein, at least that the substituted group can contain in place of one or more hydrogens a group such as alkyl, hydroxy, amino, halo, trifluoromethyl, cyano, —NH(lower alkyl), —N(lower alkyl)₂, lower alkoxy, lower alkylthio, or carboxy, and thus embraces the terms haloalkyl, alkoxy, fluorobenzyl, and the sulfur and phosphorous containing substitutions referred to below. In an embodiment, “substituted” refer to at least the substituted group can contain in place of one or more hydrogens a group such as halo or C1 to C3 alkyl group.

As used herein, “alkyl” or “alkyl group” refers to a saturated aliphatic hydrocarbon radical which can be straight or branched, having 1 to 20 carbon atoms, wherein the stated range of carbon atoms includes each intervening integer individually, as well as sub-ranges. Unless stated otherwise, “alkyl” or “alkyl group” includes substituted and unsubstituted alkyls. Examples of alkyl include, but are not limited to methyl, ethyl, n-propyl, i-propyl, n-butyl, s-butyl, t-butyl, n-pentyl, and s-pentyl. The term “lower alkyl” means an alkyl group having less than 10 carbon atoms.

As used herein, “alkenyl” or “alkenyl group” refers to an aliphatic hydrocarbon radical which can be straight or branched, containing at least one carbon-carbon double bond, having 2 to 20 carbon atoms, wherein the stated range of carbon atoms includes each intervening integer individually, as well as sub-ranges. Unless stated otherwise, “alkenyl” includes substituted and unsubstituted alkenyls. Examples of alkenyl groups include, but are not limited to, ethenyl, propenyl, n-butenyl, i-butenyl, 3-methylbut-2-enyl, n-pentenyl, heptenyl, octenyl, decenyl, and the like.

As used herein, “halo”, “halogen”, or “halogen radical” refers to a fluorine, chlorine, bromine, and iodine, and radicals thereof. Further, when used in compound words, such as “haloalkyl” or “haloalkenyl”, “halo” refers to an alkyl or alkenyl radical in which one or more hydrogens are substituted by halogen radicals. Examples of haloalkyl include, but are not limited to, trifluoromethyl, trichloromethyl, pentafluoroethyl, and pentachloroethyl.

The term “cycloalkyl” refers to a non-aromatic mono- or multicyclic ring system of about 3 to about 10 carbon atoms, preferably of about 5 to about 10 carbon atoms. Preferred ring sizes of rings of the ring system include about 5 to about 6 ring atoms. Unless stated otherwise, “cycloalkyl” includes substituted and unsubstituted cycloalkyls. Exemplary monocyclic cycloalkyl include cyclopentyl, cyclohexyl, cycloheptyl, and the like. Exemplary multicyclic cycloalkyl include 1-decalin, norbornyl, adamant-(1- or 2-)yl, and the like.

The term “aryl” as used herein, refers to an aromatic monocyclic or multicyclic ring system (fused rings). Unless stated otherwise, “aryl” includes substituted and unsubstituted aryls such as substituted and unsubstituted phenyls. Exemplary aryl groups include phenyl or naphthyl, or phenyl substituted or naphthyl substituted.

The term “heteroaryl” is used herein to denote an aromatic ring or fused ring structure of carbon atoms with one or more non-carbon atoms, such as oxygen, nitrogen, and sulfur, in the ring or in one or more of the rings in fused ring structures. Unless stated otherwise, “heteroaryl” includes substituted and unsubstituted heteroaryls. Examples are furanyl, pyranyl, thienyl, imidazyl, pyrrolyl, pyridyl, pyrazolyl, pyrazinyl, pyrimidinyl, indolyl, quinolyl, isoquinolyl, quinoxalyl, and quinazolinyl. Preferred examples are furanyl, imidazyl, pyranyl, pyrrolyl, and pyridyl.

The term “biaryl” refers to an aryl, as defined above, where two aryl groups are joined by a direct bond or through an intervening alkyl group, preferably a lower alkyl group. Unless stated otherwise, “biaryl” includes substituted and unsubstituted biaryls.

The term “fused aryl” refers to a multicyclic ring system as included in the term “aryl,” and includes aryl groups and heteroaryl groups that are condensed. Unless stated otherwise, “fused aryl” includes substituted and unsubstituted fused aryls. Examples are naphthyl, anthryl and phenanthryl. The bonds can be attached to any of the rings.

“Aralkyl” and “heteroaralkyl” refer to aryl and heteroaryl moieties, respectively, that are linked to a main structure by an intervening alkyl group, e.g., containing one or more methylene groups. Unless stated otherwise, “aralkyl” and “heteroaralkyl” includes substituted and unsubstituted aralkyls or heteroaralkyls, respectively.

The term “unit dosage form,” as used herein, refers to physically discrete units suitable as unitary dosages for human and/or animal subjects, each unit containing a predetermined quantity of a compound (e.g., compositions or pharmaceutical compositions, as described herein) calculated in an amount sufficient to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the subject.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used in the specification and claims includes one and more such excipients, diluents, carriers, and adjuvants.

As used herein, a “pharmaceutical composition” is meant to encompass a composition or pharmaceutical composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

The term “therapeutically effective amount” as used herein refers to that amount of an embodiment of the composition or pharmaceutical composition being administered that will relieve to some extent one or more of the symptoms of the condition, i.e., infection, being treated, and/or that amount that will prevent, to some extent, one or more of the symptoms of the condition, i.e., infection, that the subject being treated has or is at risk of developing.

“Pharmaceutically acceptable salt” refers to those salts that retain the biological effectiveness and optionally other properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

In the event that embodiments of the disclosed compounds in the composition or pharmaceutical composition form salts, these salts are within the scope of the present disclosure. Reference to a compound used in the composition or pharmaceutical composition of any of the formulas herein is understood to include reference to salts thereof, unless otherwise indicated. The term “salt(s)”, as employed herein, denotes acidic and/or basic salts formed with inorganic and/or organic acids and bases. In addition, when a compound contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of a compound may be formed, for example, by reacting the compound with an amount of acid or base, such as an equivalent amount, in a medium such as one in which the salt precipitates or in an aqueous medium followed by lyophilization.

Embodiments of the compounds of the composition or pharmaceutical composition of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the compounds of the composition or pharmaceutical composition of the present disclosure that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the composition or pharmaceutical composition of the present disclosure are also contemplated herein.

To the extent that the disclosed the compounds of the composition or pharmaceutical composition of the present disclosure, and salts thereof, may exist in their tautomeric form, all such tautomeric forms are contemplated herein as part of the present disclosure.

All stereoisomers of the compounds of the composition or pharmaceutical composition of the present disclosure, such as those that may exist due to asymmetric carbons on the various substituents, including enantiomeric forms (which may exist even in the absence of asymmetric carbons) and diastereomeric forms are contemplated within the scope of this disclosure. Individual stereoisomers of the compounds of the disclosure may, for example, be substantially free of other isomers, or may be admixed, for example, as racemates or with all other, or other selected, stereoisomers. The stereogenic centers of the compounds of the present disclosure can have the S or R configuration as defined by the IUPAC 1974 Recommendations.

The term “prodrug” refers to an inactive precursor of the compounds of the composition or pharmaceutical composition of the present disclosure that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

The term “administration” refers to introducing a composition of the present disclosure into a subject. One preferred route of administration of the composition is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, “treat”, “treatment”, “treating”, and the like refer to acting upon a condition (e.g., infection), a disease or a disorder with a composition to affect the condition (e.g., infection), disease or disorder by improving or altering it. The improvement or alteration may include an improvement in symptoms or an alteration in the physiologic pathways associated with the condition (e.g., infection), disease, or disorder. “Treatment,” as used herein, covers one or more treatments of an infection in a subject (e.g., a mammal, typically a human or non-human animal of veterinary interest), and includes: (a) reducing the risk of occurrence of the infection in a subject determined to be predisposed to the infection but not yet diagnosed with it (b) impeding the development of the infection, and/or (c) relieving the infection, e.g., causing regression of the infection and/or relieving one or more infection symptoms.

As used herein, the terms “prophylactically treat” or “prophylactically treating” refers completely or partially preventing (e.g., about 50% or more, about 60% or more, about 70% or more, about 80% or more, about 90% or more, about 95% or more, or about 99% or more) a condition (e.g., infection), a disease, or a symptom thereof and/or may be therapeutic in terms of a partial or complete cure for a condition (e.g., infection), a disease, and/or adverse effect attributable to the disease.

As used herein, the term “subject,” or “patient,” includes humans and mammals (e.g., mice, rats, pigs, cats, dogs, and horses), and non-mammals (e.g., ayes such as chickens etc.). Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

Host microbial organisms can be selected from, and the non-naturally occurring microbial organisms generated in, for example, bacteria, yeast, fungus or any of a variety of other microorganisms applicable to fermentation processes.

The phrase “microbial infection” can refer to a microbe colonizing the blood, a tissue and/or an organ of a subject, where the colonization causes harm to the subject. The harm can be caused directly by the microbe and/or by toxins produced by the microbe. Reference to microbe infection includes also includes microbe disease. Antimicrobial agents, such as those described herein, can kill the microbe, prevent microbe growth, and/or assist the subjects' ability to kill or prevent microbe growth.

The phrase “fungal infection” can refer to a fungus colonizing the blood, a tissue and/or an organ of a subject, where the colonization causes harm to the subject. The harm can be caused directly by the fungus and/or by toxins produced by the fungus. Reference to fungal infection includes also includes fungal disease. Antifungal agents, such as those described herein, can kill the fungus, prevent fungus growth, and/or assist the subjects' ability to kill or prevent fungus growth.

The term “fungus” can include, but is not limited to, Candida spp., (e.g., Albicans, Tropicalis, Glabrata, parapsilosis, krusei, zeylanoides, guillennondii, pelliculosa, Kefyr, dubliniensis), Epidermophyton spp., Exophiala spp., Microsporum spp., Trichophyton spp., (e.g T. rubrum and T. interdigitale), Tinea spp., Aspergillus spp., Blastomyces spp., Blastoschizoinyces spp., Coccidioides spp., Cryptococcus spp., Histoplasma spp., Paracoccidiomyces spp., Fusarium spp., Leptosphaeria spp., Mucor spp., Pneumocystis spp., spp., Saccharomyces spp., Trichoderma spp., and Trichosporon spp.

The phrase “bacterial infection” can refer to a bacteria colonizing the blood, a tissue and/or an organ of a subject, where the colonization causes harm to the subject. The harm can be caused directly by the bacteria and/or by toxins produced by the bacteria. Reference to bacterial infection includes also includes bacterial disease. Antibiotic agents, such as those described herein, can kill bacteria, prevent bacterial growth, and/or assist the subjects ability to kill or prevent bacteria growth.

Bacteria that cause bacterial infection are called pathogenic bacteria. The terms “bacteria” or “bacterium” include, but are not limited to, Gram positive and Gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The Gram-positive bacteria may include, but is not limited to, Gram positive Cocci (e.g., Streptococcus, Staphylococcus, and Enterococcus). The Gram-negative bacteria may include, but is not limited to, Gram negative rods (e.g., Bacteroidaceae, Enterobacteriaceae, Vibrionaceae, Pasteurellae and Pseudomonadaceae).

The term “antimicrobial” refers to a compound or composition that destroys antimicrobial, suppresses or prevents antimicrobial growth, and/or suppresses, prevents or eliminates the ability of the antimicrobial to reproduce.

The term “antibacterial” refers to a compound or composition that destroys bacteria, suppresses or prevents bacteria growth, and/or suppresses, prevents or eliminates the ability of bacteria to reproduce.

The term “antifungal” refers to a compound or composition that destroys fungus, suppresses or prevents fungus growth, and/or suppresses, prevents or eliminates the ability of the fungus to reproduce.

### Discussion

Embodiments of the present disclosure provide for compositions including antimicrobial agents, pharmaceutical compositions including the antimicrobial agent, methods of treatment of an infection, methods of treatment using compositions or pharmaceutical compositions, and the like. An embodiment of the present disclosure can be used to treat fungal and bacteria infections, in particular fungal infections such as those that are azole-resistant. Additional details are described below and in the Examples.

An embodiment of the present disclosure includes a composition or a pharmaceutical composition including an antimicrobial agent (e.g., antifungal, antibacterial). In an embodiment, the antimicrobial agent can be represented by the following structure:

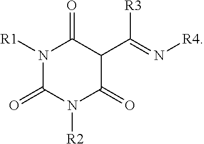

In an embodiment, R1 can be selected from: H, alkyl (e.g., methyl, ethyl, propyl), aryl (e.g., phenyl, substituted phenyl, substituted aromatic heterocycles), (CH₂)ₙCOOH with n=1-6, OH, and O-alkyl, where each alkyl and aryl group independently can be substituted or unsubstituted. In particular, R1 can be H, methyl, or phenyl group.

In an embodiment, R2 can be selected from: H, alkyl (e.g., methyl, ethyl, propyl), aryl (e.g., phenyl, substituted phenyl, substituted aromatic heterocycles), (CH₂)ₙCOOH with n=1-6, OH, and O-alkyl, where each alkyl and aryl group independently can be substituted or unsubstituted. In particular, R1 can be H, methyl, or phenyl group.

In an embodiment, R3 can be selected from: H, alkyl (e.g., methyl, ethyl, propyl), aryl (e.g., phenyl, substituted phenyl, substituted aromatic heterocycles), and COOH, where each alkyl and aryl group independently can be substituted or unsubstituted, In particular, R3 can be H, methyl, phenyl group, 4-OHC₆H₄, 1-naphthyl, 2-naphthyl, CH═CH—C₆H₅, and 4-(CH₃)₂NC₆H₄.

In an embodiment, R4 can be selected from:

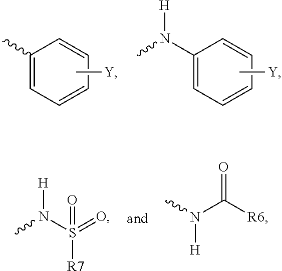

In an embodiment, Y can be selected from: H, OH, NO₂, COOH, halogen, alkyl, and O-alkly, where each alkyl group independently can be substituted or unsubstituted. In an embodiment, Y can represent multiple groups attached to the ring, where each Y group can be independently selected from the other Y group(s). In an embodiment, the Y group can be attached to the 4 position, which can be represented as 4-nitro, 4-methyl, 4-carboxy, or in 2 and 4 positions, which can represent 2,4-dinitro, 2,4-dichloro, and the like.

In an embodiment, R6 can be selected from: alkyl (e.g., methyl, ethyl, propyl), O-alkyl, O-aryl, and aryl (e.g., phenyl, substituted phenyl, substituted aromatic heterocycles), where each alkyl and aryl group independently can be substituted or unsubstituted. In particular, R6 can be methyl, phenyl, 4-HOC₆H₄, 4-O₂NC₆H₄, 4-CH₃C₆H₄, 4-CH₃OC₆H₄.

In an embodiment, R7 can be selected from: alkyl (e.g., methyl, ethyl, propyl), O-alkyl, O-aryl, and aryl (e.g., phenyl, substituted phenyl, substituted aromatic heterocycles), where each alkyl and aryl group independently can be substituted or unsubstituted. In particular, R7 can be methyl, phenyl, 4-O₂NC₆H₄, 4-CH₃C₆H₄, 4-CH₃OC₆H₄, 4-BrC₆H₄, and 1-naphthyl.

In another embodiment, the antimicrobial agent can be represented by the following structure:

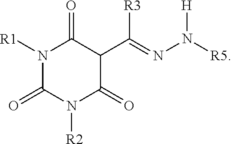

In an embodiment, R1, R2, and R3 can be groups as defined above. In an embodiment, R5 can be:

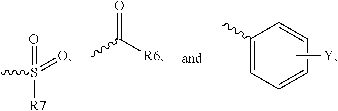

where Y, R6, and R7 can be groups as defined above.

In an embodiment, the antimicrobial agent can be represented by the following structure:

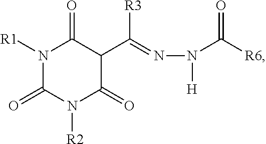

where R1, R2, R3, and R6 can be the groups as defined above.

In an embodiment, the antimicrobial agent can be represented by the following structure:

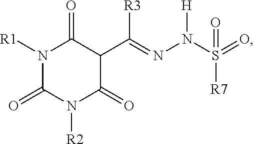

where R1, R2, R3, and R7 can be the groups as defined above.

In an embodiment, the antimicrobial agent can be made using one or more of the methods provided in Example 1. It should be noted that the reagents used in the reaction schemes shown in Example 1 can be modified by adjusting the solvents or other reactants to similar solvents or reactants that would produce the same intermediates or final products.

In an embodiment, the pharmaceutical composition includes a therapeutically effective amount of the antimicrobial agent, or a pharmaceutically acceptable salt of the antimicrobial agent, and a pharmaceutically acceptable carrier, to treat a condition (e.g., microbial infection). In an embodiment, the antimicrobial agent can include any of those described herein, in particular, those described above or pharmaceutically acceptable salts thereof, as well as prodrugs thereof.

In an embodiment, the method of treatment of an infection such as one directly or indirectly caused by a microbial infection (e.g., fungal or bacterial infection)) includes administering a therapeutically effective amount of the antimicrobial agent, or a pharmaceutically acceptable salt of the antimicrobial agent, and a pharmaceutically acceptable carrier, to treat a microbial infection. In particular, the antimicrobial agent can be used to treat fungal infections, such as azole-resistant infections.

In an embodiment the microbial infection can be caused by one or more types of microbes (e.g., one or more types of fungus and/or one or more types of bacteria). In an embodiment, the infection can be caused by an azole-resistant fungus (e.g., azole resistant Candida spp.). In an embodiment, the infection can be caused by a Candida fungus (e.g., Candida albicans and Candida glabrata) as well as others as described herein.

It should be noted that the therapeutically effective amount to result in uptake of the antimicrobial agent into the subject can depend upon a variety of factors, including for example, the age, body weight, general health, sex, and diet of the subject; the time of administration; the route of administration; the rate of excretion of the specific compound employed; the duration of the treatment; the existence of other drugs used in combination or coincidental with the specific composition employed; and like factors well known in the medical arts.

The present disclosure also provides packaged compositions or pharmaceutical compositions comprising a pharmaceutically acceptable carrier and the antimicrobial agent of the disclosure for use in treating infections. Other packaged compositions or pharmaceutical compositions provided by the present disclosure further include indicia including at least one of: instructions for using the composition to treat nicotine dependence. The kit can further include appropriate buffers and reagents known in the art for administering various combinations of the components listed above to the host.

### Pharmaceutical Formulations and Routes of Administration

Embodiments of the present disclosure include an antimicrobial agent as identified herein and can be formulated with one or more pharmaceutically acceptable excipients, diluents, carriers and/or adjuvants. In addition, embodiments of the present disclosure include an antimicrobial agent formulated with one or more pharmaceutically acceptable auxiliary substances. In particular antimicrobial agent can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the antimicrobial agent can be administered to the subject using any means capable of resulting in the desired effect. Thus, the antimicrobial agent can be incorporated into a variety of formulations for therapeutic administration. For example, the antimicrobial agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the antimicrobial agent may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the antimicrobial agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the antimicrobial agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the antimicrobial agent can be utilized in aerosol formulation to be administered via inhalation. Embodiments of the antimicrobial agent can be formulated into pressurized acceptable propellants such as dichlorodifluoromethane, propane, nitrogen and the like.

Furthermore, embodiments of the antimicrobial agent can be made into suppositories by mixing with a variety of bases such as emulsifying bases or water-soluble bases. Embodiments of the antimicrobial agent can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the antimicrobial agent in a composition as a solution in sterile water, normal saline or another pharmaceutically acceptable carrier.

Embodiments of the antimicrobial agent can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient (triamino-pyridine derivative and/or the labeled triamino-pyridine derivative) encapsulated in liposome vehicles in accordance with the present disclosure.

In an embodiment, the antimicrobial agent can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the antimicrobial agent can be accomplished using any of a variety of refillable, pump systems. Pumps provide consistent, controlled release over time. In some embodiments, the antimicrobial agent can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent (e.g., the 2,4-diaminoquinazoline compound) can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the antimicrobial agent are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the antimicrobial agent adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the antimicrobial agent may be administered using intravenous infusion, an implantable osmotic pump, a transdermal patch, liposomes, or other modes of administration. In one embodiment, a pump may be used (Sefton (1987). CRC Crit. Ref Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science249:1527-1533.

In another embodiment, the compositions of the present disclosure (as well as combination compositions separately or together) include those formed by impregnation of the antimicrobial agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the antimicrobial agent can be administered to a subject in one or more doses. Those of skill will readily appreciate that dose levels can vary as a function of the specific the antimicrobial agent administered, the severity of the symptoms and the susceptibility of the subject to side effects. Preferred dosages for a given compound are readily determinable by those of skill in the art by a variety of means.

In an embodiment, multiple doses of the antimicrobial agent are administered. The frequency of administration of the antimicrobial agent can vary depending on any of a variety of factors, e.g., severity of the symptoms, and the like. For example, in an embodiment, the antimicrobial agent can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), or four times a day. As discussed above, in an embodiment, the antimicrobial agent is administered 1 to 4 times a day over a 1 to 10 day time period.

The duration of administration of the antimicrobial agent analogue, e.g., the period of time over which the antimicrobial agent is administered, can vary, depending on any of a variety of factors, e.g., patient response, etc. For example, the antimicrobial agent in combination or separately, can be administered over a period of time of about one day to one week, about one day to two weeks.

### Routes of Administration

Embodiments of the present disclosure provide methods and compositions for the administration of the active agent (e.g., the 2,4-diaminoquinazoline compound) to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An active agent (e.g., the 2,4-diaminoquinazoline compound) can be administered in a single dose or in multiple doses.

Embodiments of the antimicrobial agent can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the 2,4-diaminoquinazoline compound. Where systemic delivery is desired, administration typically involves invasive or systemically absorbed topical or mucosal administration of pharmaceutical preparations.

In an embodiment, the antimicrobial agent can also be delivered to the subject by enteral administration. Enteral routes of administration include, but are not limited to, oral and rectal (e.g., using a suppository) delivery.

Methods of administration of the antimicrobial agent through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

### Introduction

Opportunistic fungal infections resulting from the Candida spp. represent the most common human fungal infections to date¹. Candida spp. infections can result in a broad spectrum of clinical manifestations, ranging from superficial mucocutaneous infections to the more severe invasive systemic fungal infections, and these invasive fungal infections (IFI's) are a significant cause of morbidity and mortality in at-risk populations, particularly transplant recipients, cancer patients and those infected with HIV and AIDS^{2,3}. IFI's present further diagnostic and therapeutic challenges due to the fact that they are difficult to diagnose early and are associated with high resistance rates to currently marketed antifungal agents⁴. Finally, the incidence of candidiasis caused by non-albicans Candida spp. is increasing, with Candida glabrata and Candida krusei most frequently isolated in addition to Candida albicans from clinical patients^{5,6}. Currently available clinical therapies for both cutaneous and systemic candidiasis include the first-line treatments of azoles. However, the development of clinical resistance to azoles by the Candida spp. occurs through multiple mechanisms and limits their efficacy as therapeutics. For example, resistance to azoles by Candida albicans has been shown to be largely due to overexpression of efflux pumps¹ and point mutations in ERG11 gene⁷. The opportunistic yeast pathogen Candida glabrata is also recognized for its ability to acquire resistance during prolonged treatment with azole antifungals⁸. For these reasons, there is a continuous demand for the discovery of novel therapeutics to treat fungal infections, particularly Candida spp. infections.

Hydrazine derivatives have recently begun to emerge in the literature as novel classes of antifungal agents that are proving to have therapeutic potential against numerous Candida spp., including species commonly resistant to azole antifungals. For example, it recently emerged that (4-aryl-thiazol-2-yl)hydrazines possessed potent antifungal activities against a number of clinically relevant Candida species⁹. Analogs from derivatives of the C2 and C4 positions of this hydrazine skeleton also yielded a number of promising antifungal agents that had synergistic effects with an azole, while maintaining low cytotoxicity. Finally, the hydrazine pharmacophore with substitutions of N1 together with 4-substituted phenyls at C4 of a thiazole nucleus produced a number of potent and selective hydrazine derivatives that possessed antifungal activity in the μM range⁹.

Other literature reports have shown that hydrazone derivatives have also emerged as compounds with the ability to potentiate antifungal activities in vitro. For example, the ability of hydrazone derivatives to inhibit the growth of Candida spp. was recently explored by Altintop et al¹⁰. Hydrazone derivatives bearing 5-thio-1-methyl 1H tetrazole moiety were synthesized and evaluated for potential antifungal activity and cytotoxicity, with a number of compounds showing potential for further development as antifungal agents¹⁰.

Finally, pyrimidinetrione analogs (barbituric acid derivatives) have long been explored by medicinal chemists as not only psychotropic compounds, but as anti-seizure, anticancer and antimicrobial compounds as well. For example, pyrazole and isoxazole derivatives have gained importance as potential chemotherapeutics that have applications as antimicrobials and are active against a number of different fungal species¹¹, while other pyrimidinetrione derivatives, including bisoxadiazolyl and bisthiadiazolyl pyrimidinetriones have use as antibiotic and antifungal therapies¹². In this manuscript, we present the synthesis of an extensive collection of substituted pyrimidinetrione derivatives and their biological evaluations against two clinically significant fungal pathogens from the Candida spp, namely Candida albicans and Candida glabrata. Here, we identified a number of pyrimidinetrione derivatives, including several phenylhydrazones of 5-acylpyrimidinetrione that exhibit potent antifungal activity with minimal cytotoxicity against several mammalian cell types, and provide preliminary evidence toward potential mechanisms of action for compounds with the pyrimidinetrione carbaldehyde backbone.

### Results

### Synthesis

### Preparation of 1,3-substituted 2,4,6-pyrimidinetriones

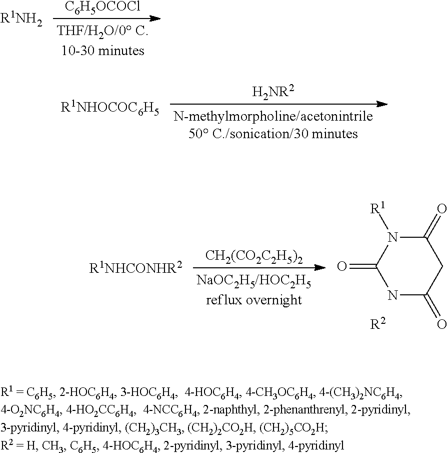

The pyrimidinetrione derivatives presented were first prepared by condensation of diethyl malonate with substituted urea in the presence of sodium ethoxide and ethanol by following the classic Dickey-Gray procedure¹³. If the appropriate substituted urea was not commercially available, then the desired substituted urea was prepared from the corresponding amines and phenyl chloroformate by following the procedure outlined in Scheme 1¹⁴. Using this method, a small library of 1,3-di and mono-substituted pyrimidinetrione derivatives were generated, and then used to further synthesize all substituted pyrimidinetrione analogs (barbituric acid analogs) presented in this work.

### Synthesis of 5-acyl-2,4,6-pyrimidinetriones

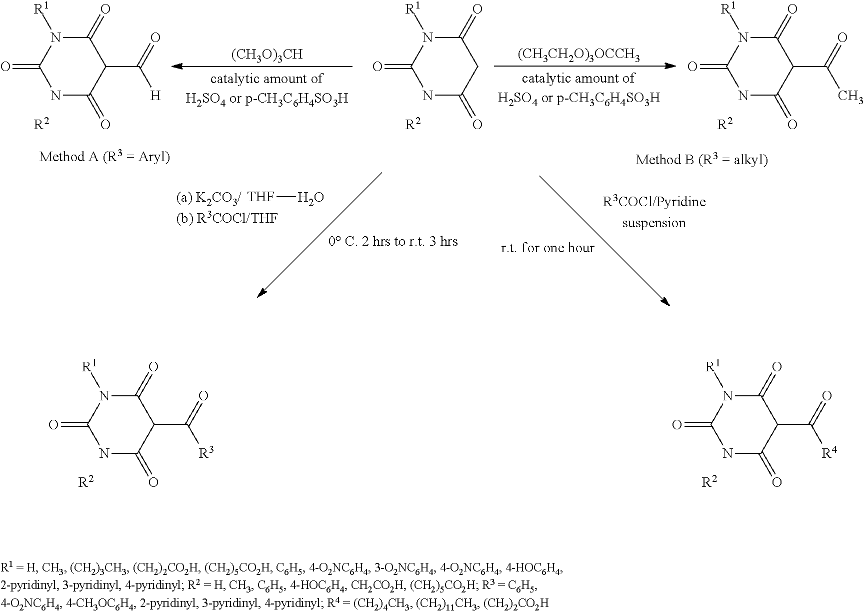

The selection of the preparation method for 5-acyl pyrimidinetriones depends on both the nature of the substituted pyrimidinetrione moiety as well as the acyl moiety. Previously, we prepared a number of derivatives, including 5-formyl-1,3-dimethylpyrimidinetrione using a modified Reimer-Tiemann reaction^{15,16}. The isolated yields using this method were <70% and furthermore, this method could not be used in the preparation of base sensitive 5-formylbarbiturates. For these reasons, we developed a new and more efficient method for preparation of 5-formyl, and 5-acetylpyrimidinetriones by using trimethyl orthoformate or triethyl orthoacetate as acylation reagents, respectively¹⁷. This method involves a simple refluxing of corresponding pyrimidinetrione in trimethyl orhoformate with catalytic amount of an acid catalyst (preferably with PTSA) for several hours (Scheme 2). For the preparation of acetyl derivatives certain reaction precautions should be taken. Namely, if the reaction is carried out with a temperature above 100° C., a substantial amount of black tar is formed and isolation of the product becomes difficult. However, if the reaction is carried out at or below 80° C. overnight, the formation of black tar is minimal and the isolated yield is almost quantitative. New methods were also developed for the preparation of 5-aroyl barbiturates (Scheme 2). The freshly prepared potassium salt of the corresponding pyrimidinetrione was condensed with aroyl chloride in THF-water as reaction media. Although this method is excellent for pyrimidinetrione acylation with aromatic acid chlorides, dismal yields were obtained with aliphatic acid chlorides due to their fast hydrolysis in the reaction media. For this reason, other acyl pyrimidinetriones were prepared in pyridine as a reaction media by following our previously published procedures¹⁸.

### Synthesis of 5-arylidene-2,4,6-pyrimidinetriones

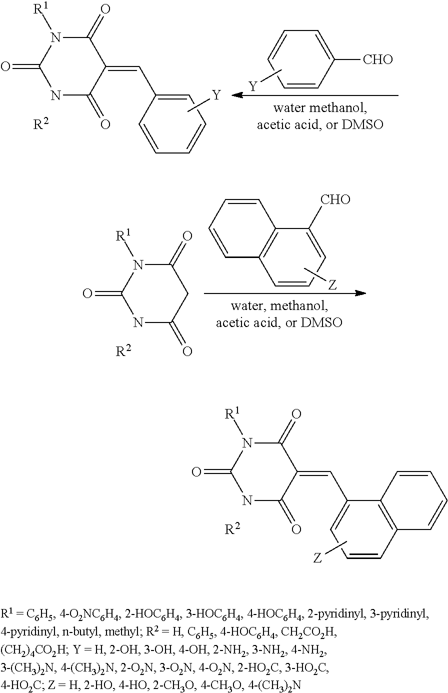

The majority of condensation products between pyrimidinetriones and aromatic aldehydes (Knoevenagel condensation) were prepared by following our previously reported procedures¹⁹. However when the condensation reaction is performed with electron rich aromatic aldehydes, such as salicylaldehyde, 4-dimethylaminobenzaldehyde, or pyridinecarbaldehyde, special precaution should be taken due to fact that two rather than one pyrimidinetrione molecule can add easily to these aldehydes²⁰. This is due to fact that the formed pyrimidinetrione α,β-conjugates (Knoevenagel condensates) are very reactive Michael acceptors²¹. One of the ways to control for the second pyrimidinetrione addition is to eliminate the Knoevenagel condensate from the reaction mixture in the course of the reaction. Encouraged by results that came out of the Deb and Bhuyan study of Knoevenagel condensation in aqueous media, we applied the same approach to the electron rich aromatic aldehydes and pyrimidinetrione derivatives in water²². The pyrimidinetriones and the electron rich aromatic aldehydes are partially soluble in water, while the Knoevenagel condensation product is noticeably less soluble. This practically eliminates the possibility of the second pyrimidinetrione addition. Using this this modification, we were successfully able to prepare a structurally diverse library of Knoevenagel condensates.

### Synthesis of 5-mono and 5,5-dialkylated-2,4,6-pyrimidinetriones

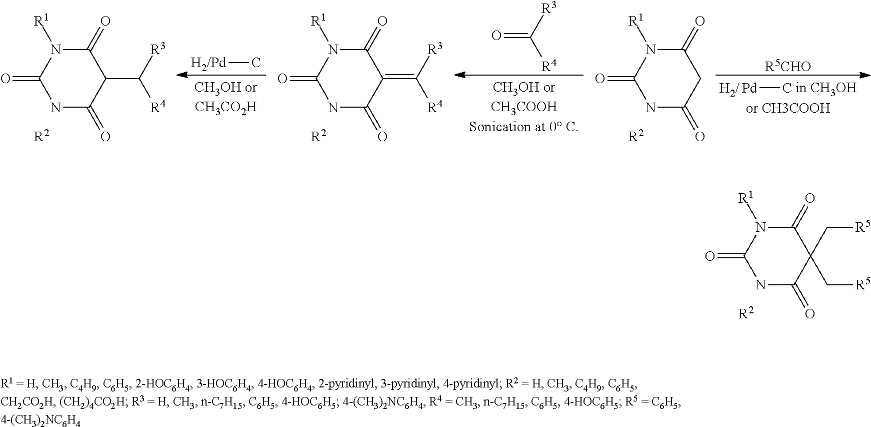

Previously, we prepared a number of these analogs using a different method of synthesis²³. Although in general the same compounds were prepared, we modified the previously reported procedures. Now, for the mono-alkylation reactions, a mixture of equivalent amounts of the corresponding benzaldehyde and 2,4,6-pyridinetrione, preferably in methanol, was sonicated at 0° C. for thirty minutes and then hydrogenation was performed with wet 10% PdC under hydrogen pressure of 30 psi. In the first step, the 5-arylidene-2,4,6-pyrimidine was prepared and without isolation, the newly formed carbon-carbon double bond was hydrogenated. By making these modifications, we avoid the double pyrimidinetrione addition to the benzaldehyde carbonyl. In the case of the dialkylated product, a mixture of the corresponding pyrimidinetrione and two equivalents of corresponding benzaldehyde were used in a similar manner and hydrogenated.

### Synthesis of Schiff Bases and Hydrazones of 5-Acyl-2,4,6-Pyrimidinetriones

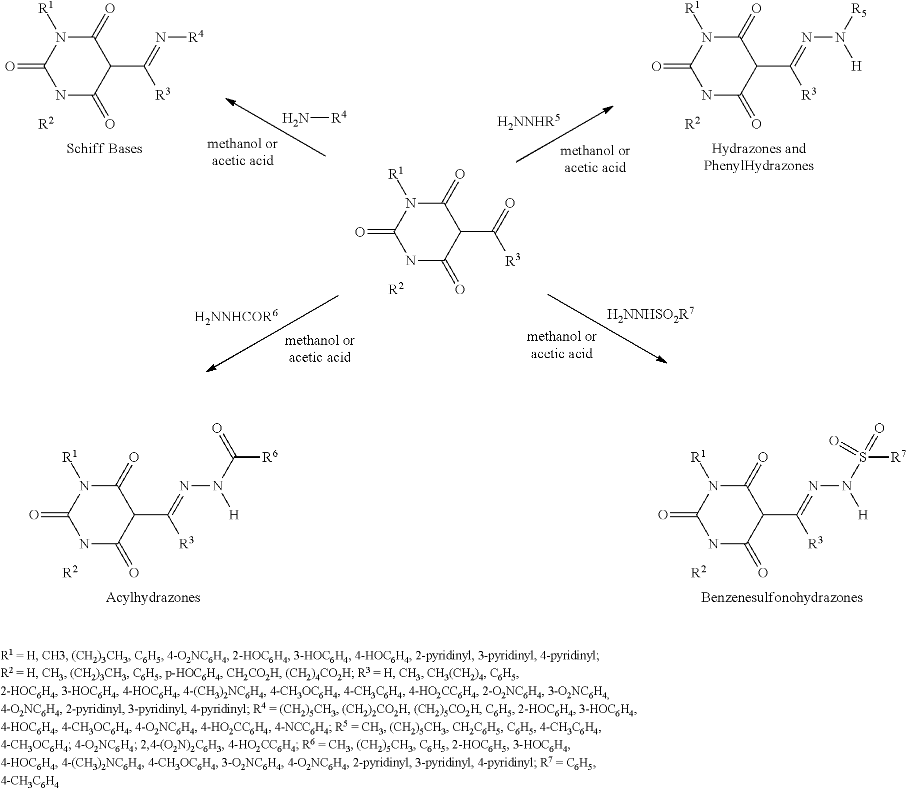

Similar reaction conditions were used for the preparation of all three classes of pyrimidinetrione derivatives: Schiff bases, hydrazones, and semicarbazones. They were all prepared from the corresponding 5-acyl pyrimidinetriones and substituted amines, hydrazines, hydrazides, or semicarbazides respectively, by following our previously published procedures²⁴. Preferably, the methanol solution or suspension was refluxed for several hours until the presence of starting materials was not detected in the reaction mixture (between three to eight hours). If one or both of the reactants were not soluble in hot methanol, then the reaction was performed in hot (˜60° C.) acetic acid. The transformations were typically quantitative and isolation procedures involved simply reducing the reaction mixture by evaporation of the solvent and crystallizing the product. Isolated yields of most of the prepared derivatives were typically >90%.

### Synthesis of Substituted Hydrazines, Hydrazides, and Benzosulfonohydrazides

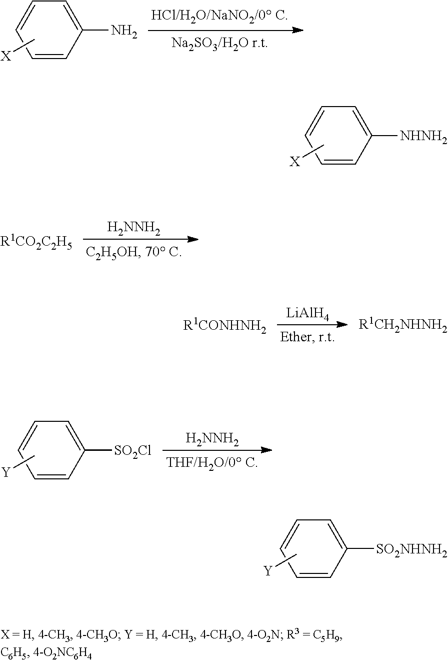

The majority of hydrazides and semicarbazides used in the preparation of the Schiff bases were not available commercially and had to be prepared. The preparation of these hydrazides started with readily available acid esters. A methanol solution of an ester with hydrazine hydrate (10 equivalents) was refluxed for one hour, followed by the distillation of methanol at atmospheric pressure. In this way, the amount of hydrazine was gradually increased to facilitate the reaction. After all the methanol was distilled from the reaction mixture, the remaining residue was mixed with water and extracted in ethyl acetate²⁵. In the case of semicarbazides, there are many different methods for the preparation of substituted semicarbazides. However, the two most commonly used are (a) from substituted urea and hydrazine hydrate²⁶ and (b) from substituted isocyanate²⁷. We have further developed a very efficient synthetic procedure for the preparation of semicarbazides that starts with the corresponding amine, phenyl chloroformate and hydrazine.²⁸

### Antifungal Activity:

The antifungal activity of the 2,4,6-pyrimidinetrione analogs shown in Schemes 2-6 were evaluated in vitro using Candida albicans (ATCC no. 10231) and Candida glabrata (ATCC no 48435). All assays were done in accordance with NCCLS reference documents²⁹. The results of these screenings are summarized in Tables 1-9 as the minimal inhibitory concentrations that inhibited more than 80% fungal growth as compared to the positive controls in 1% DMSO and RPMI media. All MIC screens were done using a visual scoring method as opposed to spectroscopic methods of analysis, due to the physical properties of many of the compounds altering their absorbance spectra relative to the positive, negative and drug controls.

Of the analogs tested, none of the 5-acyl-2,4,6-pyrimidinetriones, 5-alkylated pyrimidinetriones or the 5,5-dialkylated pyrimidinetriones showed antifungal activity through 125 μg/mL, regardless of the nature of the acyl or alkyl group attached at the 5-position of the pyrimidinetrione ring (Tables 1, 3 and 4). Alternatively, several of the 5-arylidene derivatives shown in Table 2 inhibit fungal growth of either C. albicans or C. glabrata at higher concentrations, indicating that extended conjugation of the pyrimidinetrione ring may be required for growth inhibition.

The possibility that extended conjugation of the 2,4,6-pyrimidinetrione ring may be a prerequisite for antifungal activity was an intriguing possibility. To explore this possibility more thoroughly, we synthesized and analyzed the antifungal activity of a number of Schiff base derivatives of 5-acylpyrimidinetriones, including several analogs with substituted or unsubstituted phenyl groups that would extend conjugation through the aromatic ring system. Surprisingly, of the 30 analogs synthesized, only two showed inhibitory activity of Candida albicans (compounds BA93 and BA94-Table 5). Compounds BA93 and BA94 both have a hydroxyl group in the 2-position of the aromatic ring, and the position of this hydroxyl group positions a non-bonded electron pair close to the carbon-nitrogen double bond of the molecule. In contrast, compounds with a hydroxyl in the 3-position (BA95, BA96) or in the 4-position (BA79-81, Table 5) of the aromatic ring are inactive with respect to inhibition of fungal growth. Collectively, these data indicate that the contribution from the non-bonding electron pair close to the carbon-nitrogen bond might be integral for antifungal activity. Derivatives of the 2,4,6-pyrimidinetriones that contain hydrazone moieties share similar structural and functional properties to compounds BA93 and BA94, which contain the 2-OH phenyl group with the contribution from the non-bonded electron pair to the carbon-nitrogen double bond. To test whether this structural component was important in antifungal function, hydrazones of 5-acylpyrimidinetriones and phenylhydrazones of 5-acylpyrimidinetriones synthesized in Schemes 5 and 6 were evaluated for antifungal activity. We found that in 5-acylpyrimidinetrione hydrazones that contained only a H or CH3 moiety, minimal growth inhibition was observed (Table 6 and Table 8), even with the extended conjugation provided through the C—N and N—N double bonds. However, in the cases where the substitution to 5-acylpyrimidinetriones was a phenylhydrazone, growth inhibition significantly increases, and over 80% inhibition could be observed through lower dilutions, typically in the range of 2-4 μg/mL (Table 7, BA22, BA23, BA25, BA73, BA74, BA78 and BA85). The addition of one NO2 group in the 4-position of the phenyl ring did not decrease or increase antifungal activity of 1,3-dimethyl 1,3-unsubstituted molecules, but the addition of a phenyl group to the 3-position of the pyrimidinetrione ring greatly reduced inhibition (Table 7, BA28).

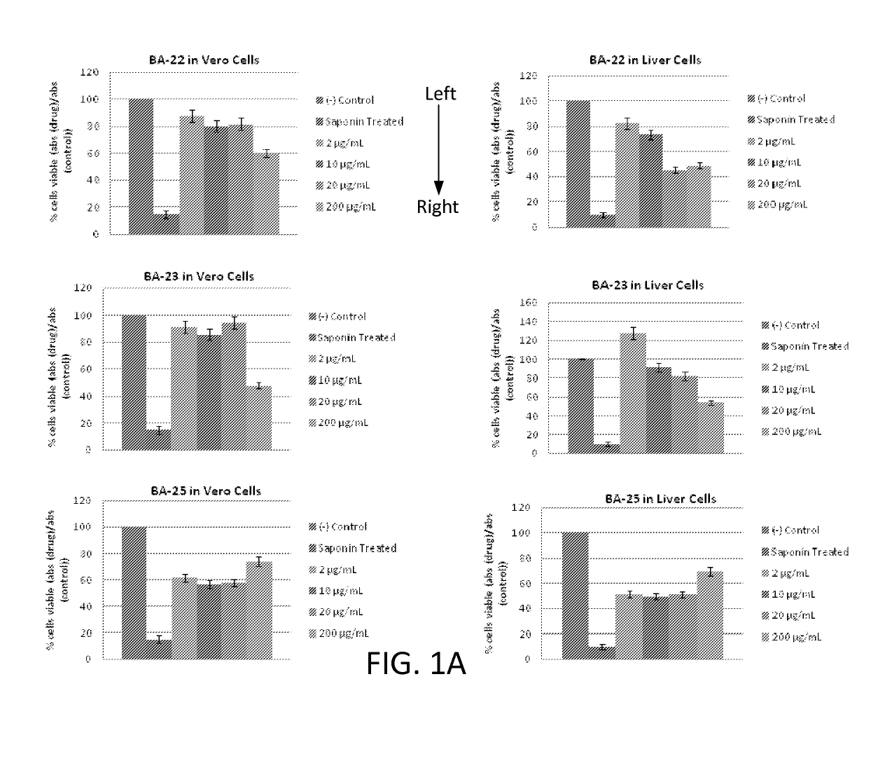

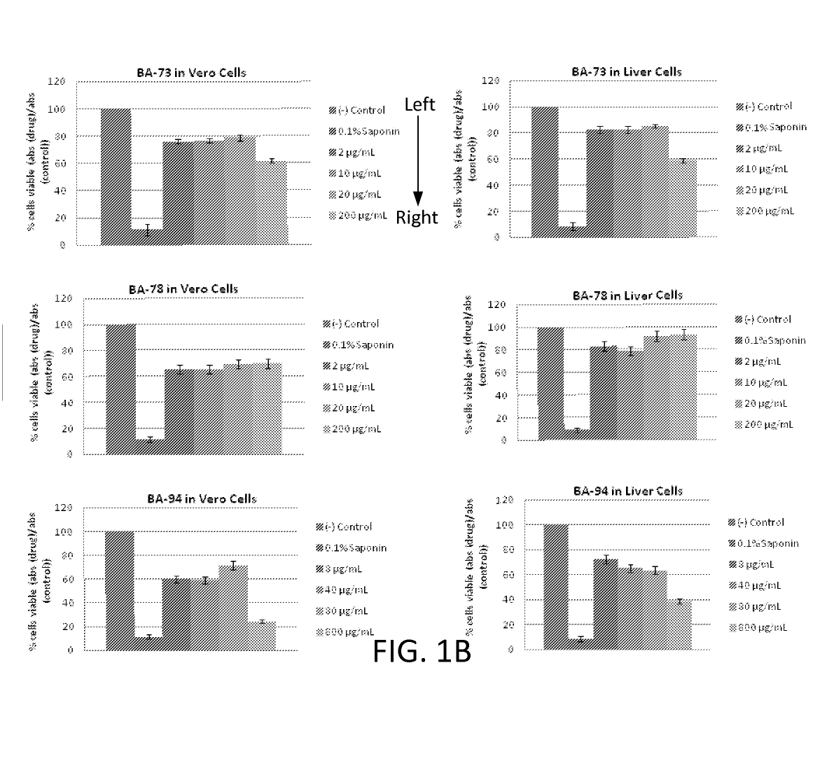

Finally, in vitro mammalian cell toxicity studies were done using both mammalian kidney cells and human liver cells (Vero (kidney) cells-ATCC no. CRL-1651 and Hep G2 (liver) ATCC no. HB-8065) on all derivatives that had antifungal activity of less than 8 μg/mL. Cytotoxicity studies were performed in accordance with Promega CellTiter 96 Non-RadioactivCell Proliferation Assay (cat # G4000). Representative compounds are presented in FIGS. 1A and 1B. The data is presented as the percent cells that remain viable, compared to the negative control, which was set to 100%. BA22, BA23 and BA73 had minimal toxicity in the kidney cells, with over 80% cells viable even at a concentration 10 times higher than the MIC (Table 7). However, compounds BA25, BA78 and BA94 all had a reduction of ˜35% in cell viability, indicating that these three compounds may be more cytotoxic, at least to cells from the kidney origin. Similar results were observed with respect to the cell viability assays using hepatocytes (FIGS. 1A and 1B).

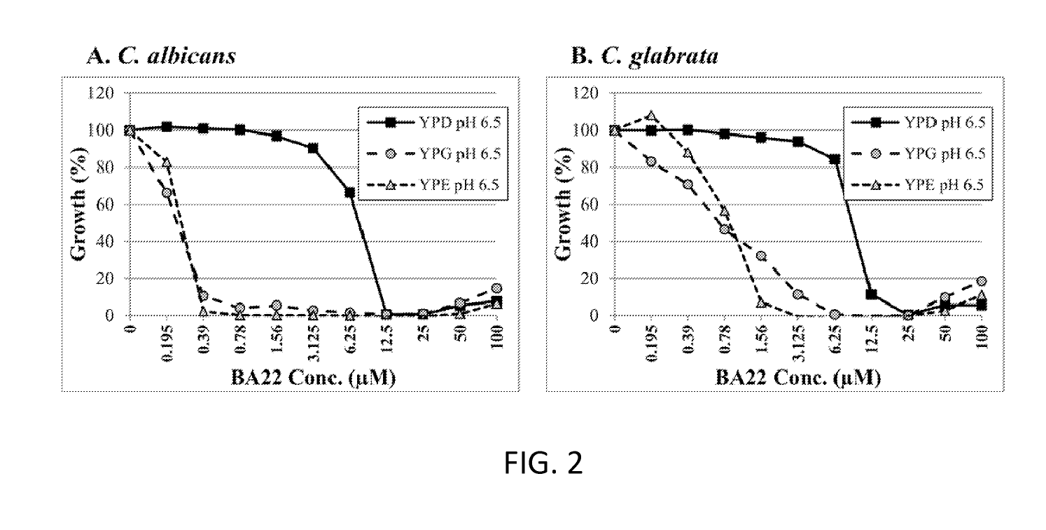

Considering the significant growth inhibition and lack of overall lack of cytotoxicity of many of the phenylhydrazones, we further explored potential mechanisms of action using the compound BA22 as a representative drug. We initially observed that in the presence of BA22, C. albicans formed small colonies similar to respiration deficient ‘petite’ mutants previously described for Saccharomyces. We observed that on agar plates enriched with a range of BA22 concentrations, C. albicans colony formation was significantly delayed in a dose dependent manner, eventually forming small colonies (data not shown). This phenotype somewhat resembles that of respiration deficient ‘petite’ mutants, which are unable to utilize non-fermentable carbon sources such as glycerol and ethanol³⁰. We therefore determined if BA22 affected the ability of C. albicans or C. glabrata to use glycerol or ethanol as carbon sources. The concentrations of BA22 required to inhibit the growth of either fungi were much lower when glycerol or ethanol is provided as a carbon source versus glucose (FIG. 2). This suggests that BA22 may preferentially interfere with the utilization of non-fermentable carbon sources, and thus may cause a defect in respiration. Growth inhibition occurred at higher BA22 concentrations in glucose medium, possibly indicating an additional secondary mechanism.

### Discussion and Conclusions:

In this Example, we have outlined the synthetic preparations of a wide range of 2,4,6-pyrimidinetrione analogs, and their potential for inhibition of fungal growth. Of the compounds synthesized and analyzed, we found that pyrimidinetriones containing either a 2-OH phenyl moiety or a phenyl hydrazone moiety possess potent growth inhibition for Candida albicans and Candida glabrata. Based on these results hypothesize that extension of the 2,4,6-pyrimidinetrione ring conjugation, either from the non-bonded electron pair of the 2-OH group or the phenyl hydrazone moiety is essential for antifungal activity in pyrimidinetrione analogs.

We initially observed that in the presence of BA22, C. albicans formed small colonies similar to respiration deficient ‘petite’ mutants previously described for Saccharomyces. Thus the severe growth inhibition caused by BA22 could indicate a defect in energy production. Consistent with this argument, BA22 inhibits fungal growth at significantly lower concentrations when non-fermentable carbon sources such as ethanol and glycerol are provided as compared to glucose, suggesting that phenylhydrazones of pyrimidinetriones may cause defects in respiration. Alternatively, it is possible that when energy production is dependent upon respiration, both C. albicans and C. glabrata are somehow sensitized to the effects of BA22. Either way, at higher concentrations growth in the presence of glucose was also inhibited, indicating a potential secondary mechanism of growth inhibition. More comprehensive mechanistic studies are currently underway that will more precisely define the effects of BA22 on fungal energy production.

### Experimental Section

### Preparation of Diluted Compounds

None of the compounds tested over the course of these studies were soluble in water and DMSO was used to dilute each compound tested. Master stock concentrations of all tested compounds were prepared to ensure that the maximum final concentration of DMSO was 1% or less. Subsequent 2-fold serial dilutions were made using sterile water to a final concentration of 1280-5.00 μg/ml. A final 10-fold dilution of each drug was made by aliquotting 0.1 mL of each drug dilution into 0.9 mL of fungal suspension in RPMI 1640 media with 10 mM HEPES buffer added (see methodology below), giving final concentrations tested in the range of 128-0.5 μg/ml.

### MIC Determination:

Antifungal susceptibility studies and minimal inhibitory concentrations (MIC) values for Candida albicans (ATCC no. 10231) and Candida glabrata (ATCC no. 48435) were determined by the broth microdilution technique in accordance with NCCLS reference documents. Microdilution panels ranged from 0.5 to 128 μg/mL. All organisms were subcultured on Sabouraud agar and passaged to ensure purity and viability. The initiating inocula were prepared by picking 5-7 isolated colonies ˜1 mm in diameter from freshly prepared YM agar plates. Colonies were resuspended in sterile water, vortexed and the cell density was adjusted spectrophotometrically to the transmittance of a 0.5 McFarland standard at a wavelength of 530 nm, to yield a stock suspension of 1×10⁶-5×10⁶ cells/mL. The stock was allowed to hydrate for 30 minutes at room temperature. A working suspension was then made by diluting the stock 1:50 with RPMI 1640 media containing HEPES buffer. The working suspension was used for all assays. The plates were incubated at 35° C. for 48-72 hours in a humid atmosphere. Growth was scored visually. MIC values are defined as the lowest concentration of agent that prevents any discernible growth, ˜80% reduction of growth, as compared with drug-free control wells. All assays were run in parallel using either fluconazole or amphotericin B at the NCCLS recommended concentrations²⁹ as a drug control (Sigma-Aldrich).

### In Vitro Mammalian Cell Toxicity Assays:

Cytotoxicity was determined using non-cancerous Vero cells (African green monkey kidney cells-ATCC no. CRL-1651) and Hep G2 (liver) (ATCC no. HB-8065) cells in accordance with Promega CellTiter 96 Non-RadioactivCell Proliferation Assay (cat # G4000). Cells were grown for 24 h at 37° C. and 5% CO₂ in a 96-well culture plate. All compounds with MIC≦8 μg/ml were further screened in the MTT assays using both cell types. Compounds that fit these criteria were diluted in media to the testing dilution amounts: the MIC concentration (shown in respective tables; 5×MIC; 10×MIC and 100×MIC. The media used to grow cells was DMEM containing 10% FBS and 2%. Cells were incubated in the presence of the compounds for 24 hours at 37° C. and 5% CO₂. Tetrazolium dye solution was added to each well and allowed to incubate for 1-4 h. Stabilization/Stop solution was added and allowed to sit at room temperature for 1 h. Formazan product was scored spectrophotometrically with an automatic plate reader set at 570 nm. 0.1% saponin was used as a positive control. 1% DMSO controls were also used since the compounds were reconstituted in DMSO.

### Testing for Respiratory Deficiencies:

Respiratory deficiencies were examined using YPD (2% glucose), YPE (3% ethanol), or YPG (3% glycerol)³¹. Each medium was buffered with 0.3 M MOPS, and pH adjusted to 6.5 with KOH. Serial 1:1 dilutions of BA22 were performed in DMSO from a 20 mM stock. Each drug stock or DMSO alone was then diluted further 1:99 with each of the above media. Medium+drug suspensions (100 μl) were then transferred to a round bottomed 96 well plate. C. albicans strain SC5314³² and C. glabrata strain CS 177.93³³, grown overnight in YPD broth at 30° C. (180 rpm) were washed in distilled water, and resuspended at 1×10⁴ cells ml⁻¹ in each of the above three media. Then 100 μl of each cell suspension (approx. 10³ cells) was added to wells of the 96 well plate containing the corresponding medium+drug combinations. Final DMSO concentration was 0.5% in all wells, with final drug concentrations of 0 and 0.195-100 μM. After 24 and 48 hours incubation at 30° C., growth was quantified by measuring OD₆₀₀ₙₘ using a BioTek plate reader. Growth at each BA concentration was then expressed as % growth vs. the DMSO control of the same medium.

### Chemistry Experimental:

Thin-layer chromatographic analysis (TLC) was performed using silica gel on aluminum foil glass plates and products were detected under ultraviolet (UV) light. The ¹H and ¹³C NMR spectra were run on Varian 400 MHz Unity instruments in CDCl₃ or in DMSO-d₆ with solvent signals as internal standards. When necessary, products were purified by flash chromatography on silica gel (40-70 mm) from Sorbent Technologies. Hydroxyphenylhydrazines were prepared from corresponding aminophenol by following preparation procedure³⁴ and/or following the procedure for preparation of 3-hydroxyphenylhydrazine [Mao, J.; Wu, G.; Chen, M.; Gu, Z.; Qian, Y.; Yuan, Z.; Luo, B. “Process for p reparation of hydroxy and alkoxy substituted phenylhydrazines” Farming Zhuanli Shenqing Gongkai Shuomingshu, 101602690, 16 Dec. 2009.] All reagents and solvents were purchased from Sigma-Aldrich and were analytical grade.

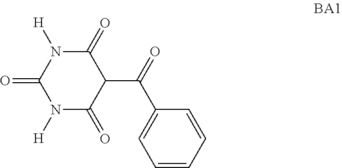 [SMILES: O=C1NC(=O)C(C(=O)c2ccccc2)C(=O)N1]

### Typical Procedure for Preparation of N-unsubstituted 5-Aroyl-2,4,6-pyrimidinetriones

Preparation of 5-benzoyl-pyrimidine-2,4,6-trione (BA1). Pyridine (100 ml) suspension of barbituric acid (2.56 g; 0.02 mol) was heated at 70 C until dissolves. Into this dark yellow solution at 70 C benzoyl chloride was slowly added (2.8 g; 0.02 mol). Immediately color changed to read and then to dark read solution. Stirring at 70° C. continue for additional two hours, and then at room temperature overnight. Solvent was evaporated under reduce pressure. Dark solid was dissolved in hot water (400 ml) and acidified with concentrated hydrochloric acid to pH˜2 and left at room temperature for two hours. Dark read solid was separated by filtration, washed with water (5×100 ml) and dried at 110 C for two hours to give pure product (4.1 g; 89%). ¹H-NMR (DMSO-d₆) δ 11.47 (2H, NH), 7.55 (2H, d, J=7.6 Hz, o-H), 5.55 (1H, t, J=7.2 Hz, p-H), and 7.41 (2H, t, J=7.6 Hz) ppm. ¹³C-NMR (DMSO-d₆) δ 190.8, 149.9, 135.8, 132.2, 129.2, 128.2, and 95.7 ppm.

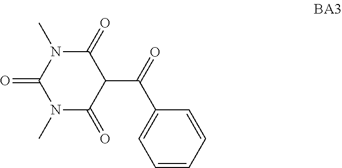 [SMILES: CN1C(=O)C(C(=O)c2ccccc2)C(=O)N(C)C1=O]

### Typical Procedure for Preparation of N-Substituted 5-Aroyl-2,4,6-pyrimidinetriones

Preparation of 5-benzoyl-1,3-dimethylpyrimidine-2,4,6-trione (BA3). A mixture of 3,3-dimethyl-2,4,6-pyrimidinetrione (1.56 g; 0.01 mol) and sodium bicarbonate (1.26 g; 1.5 .mol) was dissolved in water. Into this solution with stirring at room temperature tetrahydrofuran (60 ml) solution of benzoyl chloride (1.4 g; 0.01 mol) was added. The resulting clear reaction mixture was stirred at room temperature for one hour. The colorless reaction mixture changes from yellow to brown and finally to red, indicating the progress of product formation. The reaction volume was reduced to approximately 10 ml by evaporation of solvent. The ice water-cooled reaction mixture was acidified with 10% HCl to pH˜2. Water was decanted from the white gummy precipitate. This precipitate was washed with cold water (3×10 ml) and crystallized from ethanol to give 2.2 g (85% yield) pure product. ¹H-NMR (DMSO-d₆) δ 7.51 (3H, d+t), 7.42 (2H, d, J=8.0 Hz), and 3.17 (6H, s) ppm. ¹³C-NMR (DMSO-d₆) δ 185, 163, 146, 131, 127, 124, 123, 92, and 24 ppm.

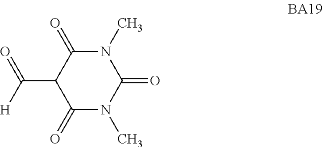

### Typical procedure for 5-formylation of 2,4,6-pyrimidinetriones with trimethyl orthoformate

Preparation of 1,3-dimethyl-2,4,6-trioxohexahydropyrimidine-5-carbaldehyde (BA19). White suspension of trimethyl orthoformate (300 ml), 1,3-dimethyl-2,4,6-pyrimidinetrione (50 g; 0.32 mol), and sulfuric acid (two drops) was stirred with refluxing for five hours. After approximately ten minutes, the reaction mixture transformed into a clear yellow solution that shortly turns again into yellow suspension. The reaction suspension was left at room temperature for several hours. Solid product was separated by filtration, washed with ether (3×30 ml), and dried on air to give pure product (51 g; 86%). ¹H-NMR (CDCl₃) δ 9.25 (1H, s, CH), 8.64 (1H, s, 5-H), 3.35 (3H, s, NCH₃), and 3.33 (3H, s, NCH₃) ppm. ¹³C-NMR (CDCl₃) δ 178.2, 156.2, 154.4, 101.0, 28.2 and 27.6 ppm; MS-EI, m/z 184 (M⁺, 20%), 169 ((M-CH₃)⁺, 28%), 156 (M-CO)⁺, 39%).

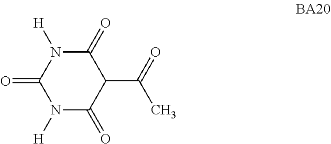

### Typical procedure for 5-acetylation of 2,4,6-pyrimidinetriones with triethyl orthoacetate

Preparation of 5-acetyl-1,3-dimethylpyrimidine-2,4,6-trione (BA20). Triethyl orthoacetate (20 ml) suspension of 2,4,6-pyrimidinetrione (2.56 g; 0.02 mol), and p-toluenesulfonic acid monohydrate (200 mg) was heated at 80° C. overnight. Reaction mixture was suspension all the time. Reaction mixture was concentrated to about ⅓ of the original volume and left at room temperature overnight. Solid material was separated by filtration from the dark solution, washed with a small portion of ice-water chilled acetone, ether (3×10 ml) and dried on air to give pure product (2.9 g; 85%). ¹H-NMR (DMSO-d₆) δ 11.77 (1H, s, NH), 11.04 (1H, s, NH), and 2.56 (3H, s, CH₃) ppm. ¹³C-NMR (DMSO-d₆) δ 195.67, 149.8, 96.1, and 24.5 ppm. Elemental Analysis. Calc: C, 42.36; H, 3.55; N, 16.47. Fund: C, 42.25; H, 3.68; N, 16.35.

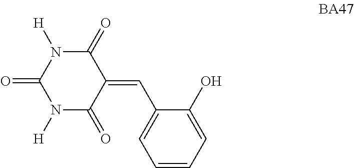 [SMILES: O=C1NC(=O)C(=Cc2ccccc2O)C(=O)N1]

### Typical procedure for preparation of 5-rylidene-2,4,6-pyrimidinetrione

Preparation of 5-(2-hydroxybenzylidene)pyrimidine-2,4,6-trione (BA47). Salicylic aldehyde (1.22 g; 0.01 mol) was added into a stirring water (75 ml) solution of 2,4,6-pyrimidinetrione (1.28 g; 0.01 mol). Immediately after mixing, an orange precipitate starts to form. The resulting orange suspension was stirred at room temperature for additional 10 minutes. The solid product was separated by filtration, washed with water (3×10 ml) and dried on air to give 2.2 g (91%) of pure product. ¹H-NMR (DMSO-d₆) δ 11.29 (1H, s), 11.12 (1H, s), 10.58 (1H, s), 8.60 (1H, s), 8.14 (1H, d, J=8.0 Hz), 7.35 (1H, t, J=8.0 Hz), 6.91 (1H, d, J=8.4 Hz), 6.81 (1H, t, J=8.0 Hz) ppm. ¹³C-NMR (DMSO-d₆) δ 164.4, 162.5, 159.7, 151.0, 135.4, 133.5, 120.6, 118.9, 117.8, and 116.1 ppm.

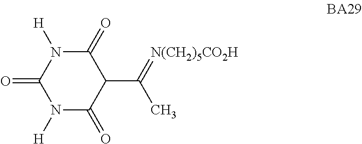

### Typical Procedure for Preparation of Schiff Bases with Amino Acids

Preparation of 6-{(E)-[1-(2,4,6-trioxohexahydropyrimidin-5-yl)ethylidene]amino}hexanoic acid (BA29). Methanol (500 ml) suspension of 5-acetyl-2,4,6-pyrimidinetrione (1.7 g; 0.01 mol) and 5-aminohexanoic acid (1.3 g; 0.01 mol) with kaolin clay (1 g) was refluxed with stirring overnight. Insoluble material was separated by hot filtration. Filtrate volume was reduced to ˜30 ml and cooled at ice water for thirty minutes. Formed white powder was separated by filtration, washed with ether (3×10 ml) and dried on air to give pure product (2.1 g; 74%).

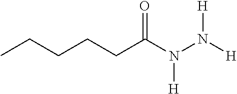

### Typical Procedure for Preparation of Hydrazides

Preparation of Hexanehydrazide. A methanol solution (50 ml) of hydrazine hydrate (5 g; 0.1 mol) and methyl hexanoate (1.3 g; 0.01 mol) was refluxed for 90 minutes. Methanol was distilled off at atmospheric pressure and the resulting oily residue was mixed with ethyl acetate (200 ml) and water (50 ml). The ethyl acetate layer was separated, washed with water (3×10 ml), and dried over anhydrous sodium sulfate. Evaporation of ethyl acetate yields a white solid product in 93% isolated yield. ¹H-NMR (CDCl₃) δ 6.98 (1H, s, NH), 3.91 (2H, s, NH₂), 2.13 (2H, t, J=7.6 Hz, COCH₂), 1.62 (2H, q, J=7.6 Hz, COCH₂CH₂), 1.29 (4H, m, CH₂CH₂), and 0.88 (3H, t, J=7.6 Hz, CH₃) ppm. ¹³C-NMR (CDCl₃) δ 174.4, 34.7, 31.6, 25.4, 22.6, and 14.1 ppm.

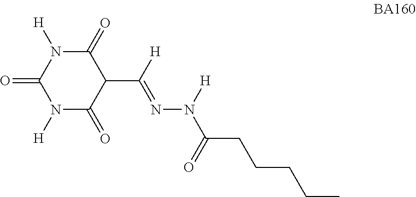 [SMILES: CCCCCC(=O)N/N=C/C1C(=O)NC(=O)NC1=O]

Typical procedure for preparation non-substituted barbituric acid hydrazones: Preparation N′-[(E)-(2,4,6-trioxohexahydropyrimidin-5-yl)methylidene]hexanehydrazide (BA160). Methanol (1 L) suspension of 5-formylbarbituric acid (780 mg, 5 mmol) and hexanehydrazide (650 mg; 5 mmol) was refluxed for two hours and concentrated to volume of about 100 ml. Still hot methanol suspension was filtered and white solid product was washed with methanol (3×10 ml) and dried at 110° C. for 10 minutes to give 1.2 g (90%) white crystalline product. ¹H-NMR (DMSO-d₆) δ 11.09 (1H, s, NH), 10.78 (1H, s, NH), 10.67 (1H, s, NH), 7.90 (1H, s, CH), 2.13 (2H, t, J=7.2 Hz, COCH₂), 1.50 (2H, m, COCH₂CH₂), 1.22 (4H, m, CH₂CH₂), and 0.82 (3H, t, J=6.4 Hz, CH₃) ppm. ¹³C-NMR (DMSO-d₆) δ 171.4, 166.0, 164.2, 156.4, 151.5, 89.9, 33.7, 31.4, 24.9, 22.5, and 14.4 ppm.

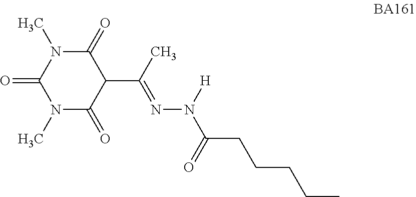 [SMILES: CCCCCC(=O)N/N=C(\C)C1C(=O)N(C)C(=O)N(C)C1=O]

Typical procedure for preparation of multiple substituted barbituric hydrazones. Preparation of N′-[(1E)-1-(1,3-dimethyl-2,4,6-trioxohexahydropyrimidin-5-yl)ethylidene]hexanehydrazide (BA161). Methanol (200 ml) solution of 5-acetyl-1,3-dimethylbarbituric acid (990 mg; 5 mmol) and hexanehydrazide (650 mg; 5 mmol) was refluxed for 2 hours. Reaction mixture was concentrated to 10 ml and diluted with ether (100 ml). Resulting solution was left at room temperature for two hours. Formed white crystalline product was separated by filtration, washed with ether (3×10 ml) and dried on air to give 1.45 g (94%) of pure product. ¹H-NMR (CDCl₃), δ 13.61 (1H, s, CH), 9.10 (1H, s, NH), 3.23 (3H, s, NCH₃), 3.21 (3H, s, NCH₃), 2.59 (3H, s, CH₃), 2.30 (2H, t, J=7.6 Hz, COCH₂), 1.65 (2H, q, J=7.6 Hz, COCCH₂), 1.29 (4H, m), and 0.87 (3H, t, J=6.8 Hz, CH₃) ppm. ¹³C-NMR (CDCl₃) δ 174.3, 172.4, 166.1, 162.6, 151.4, 90.3, 34.2, 31.5, 28.2, 27.9, 25.2, 22.5, 16.7, and 14.1 ppm.

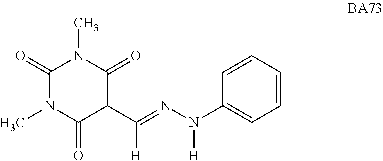 [SMILES: CN1C(=O)C(/C=N/Nc2ccccc2)C(=O)N(C)C1=O]

### Typical procedure for preparation of phenylhydrazones of 5-Acyl-2,4,6-pyrimidinetrione derivatives

Preparation of 1,3-dimethyl-5-[(E)-(2-phenylhydrazinylidene)methyl]pyrimidine-2,4,6-trione (BA73). A methanol solution (100 ml) of carbaldehyde (1.82 g; 0.01 mol) and phenylhydrazine (1.3 g; 0.012 mol) was refluxed for two hours. The reaction mixture was left at room temperature overnight and cooled in ice water for 10 minutes before being filtered. The solid product was washed with ice-water cooled methanol (3×10 ml) and dried on air to give pure product in 95% (2.6 g) isolated yield. ¹H-NMR (DMSO-d₆) δ 11.15 (1H, d, J=11 Hz, NH), 8.79 (1H, s), 8.14 (1H, d, J=11 Hz), 7.19 (2H, t, J=8 Hz), 6.83 (1H, t, J=7 Hz), 6.73 (2H, d, J=8 Hz) 3.10 (3H, s), and 3.09 ppm (3H, s); ¹³C-NMR (DMSO-d₆) δ 163.9, 162.6, 160.8, 158.3, 152.1, 148.4, 129.7, 121.3, 113.7, 90.3, 28.1, and 27.5 ppm.

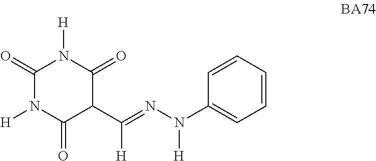 [SMILES: O=C1NC(=O)C(/C=N/Nc2ccccc2)C(=O)N1]

5-[(E)-(2-phenylhydrazinylidene)methyl]pyrimidine-2,4,6-trione (BA74). Isolated yield 92%. ¹H-NMR (DMSO-d₆) δ 11.08 (1H, d, J=6.8 Hz), 10.80 (1H, s), 10.70 (1H, s), 8.72 (1H, s), 8.02 (1H, d, J=6.8 Hz), 7.22 (2H, t, J=8.0 Hz), 6.86 (1H, t, J=7.6 Hz), and 6.74 (2H, d, J=8.0 Hz) ppm. ¹³C-NMR (DMSO-d₆) δ 166.1, 164.3, 160.1, 151.6, 148.5, 129.8, 121.4, 113.9, and 90.2 ppm.

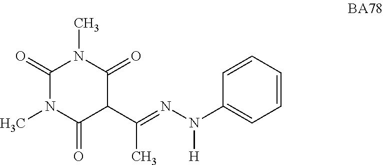 [SMILES: C/C(=N\Nc1ccccc1)C1C(=O)N(C)C(=O)N(C)C1=O]

1,3-dimethyl-5-[(1E)-1-(2-phenylhydrazinylidene)ethyl]pyrimidine-2,4,6-trione (BA78). Isolated yield 93%. ¹H-NMR (CDCl₃) δ 13.47 (1H, s), 7.26 (2H, t, J=8.0 Hz), 6.96 (1H, t, J=7.6 Hz), 6.77 (2H, t, J=8.0 Hz), 6.34 (1H, s), 3.29 (6H, s), and 2.81 (3H, s) ppm. ¹³C-NMR (CDCl₃) δ 177.5, 166.4, 163.0, 151.6, 145.9, 129.9, 122.4, 113.5, 90.1, 28.2, 28.0, and 16.8 ppm.

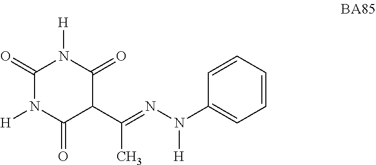 [SMILES: C/C(=N\Nc1ccccc1)C1C(=O)NC(=O)NC1=O]

5-[(1E)-1-(2-phenylhydrazinylidene)ethyl]pyrimidine-2,4,6-trione (BA85). Isolated yield 89%. ¹H-NMR (DMSO-d₆) δ 13.25 (1H, s), 10.64 (2H, broad s), 8.53 (1H, s), 7.24 (2H, t, J=8.0 Hz), 6.87 (1H, t, J=7.2 Hz), 6, 74 (2H, d, J=8.0 Hz), and 2.65 (3H, s). ¹³C-NMR (DMSO-d₆) δ 175.6, 150.5, 147.3, 130.1, 121.4, 113.4, 88.9, and 16.4 ppm.

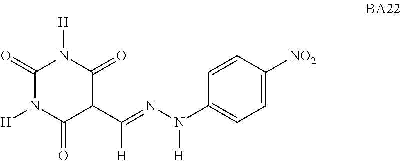 [SMILES: O=C1NC(=O)C(/C=N/Nc2ccc([N+](=O)[O-])cc2)C(=O)N1]

### Typical procedure for preparation of hydrazones of 2,4,6-pyrimidinetriones

Preparation of 5-{(E)-[2-(4-nitrophenyl)hydrazinylidene]methyl}pyrimidine-2,4,6-trione (BA22). A hot methanol (200 ml) solution of 2,4,6-trioxohexahydropyrimidine-5-carbaldehyde (1.56 g, 0.01 mol) and a methanol solution (200 ml) of 4-nitrophenylhydrazine were mixed together and refluxed with stirring for two hours. The still hot reaction mixture was filtered. A dark brown solid was discarded and the filtrate was reduced (˜75 ml). The formed yellow precipitate was separated by filtration, washed with cold methanol (3×15 ml) and dried on air to give pure product (2.2 g; 76%). ¹H-NMR (DMSO-d₆) δ 11.25 (1H, broad singlet), 10.85 (1H, s), 10.75 (1H, s), 9.86 (1H, s), 8.11 (2H, d, J=9.2 Hz), 6.80 (2H, d, J=9.2 Hz) ppm. ¹³C-NMR (DMSO-d₆) δ 165.8, 164.2, 159.8, 154.3, 151.5, 140.3, 126.5, 112.5, and 91.3 ppm.

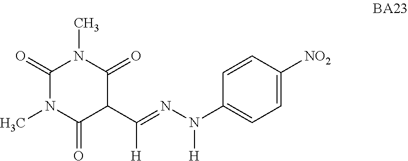 [SMILES: CN1C(=O)C(/C=N/Nc2ccc([N+](=O)[O-])cc2)C(=O)N(C)C1=O]

1,3-Dimethyl-5-{(E)-[2-(4-nitrophenyl)hydrazinylidene]methyl}pyrimidine-2,4,6-trione (BA23). Isolated yield 87%. ¹H-NMR (DMSO-d₆) δ 11.34 (1H, d, J=7.6 Hz), 9.93 (1H, s), 8.09 (3H, d, J=8.4 Hz), 6.79 (2H, d, J=8.4), 3.13 (3H, s), and 3.09 (3H, s) ppm. ¹³C-NMR (DMSO-d₆) δ 163.6, 162.5, 160.5, 154.2, 152.1, 140.2, 126.4, 112.3, 91.3, 28.2, and 27.5 ppm.

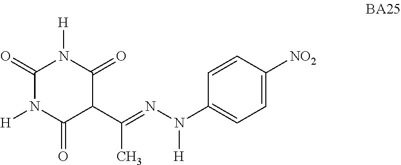 [SMILES: C/C(=N\Nc1ccc([N+](=O)[O-])cc1)C1C(=O)NC(=O)NC1=O]

5-{(1E)-1-[2-(4-Nitrophenyl)hydrazinylidene]ethyl}pyrimidine-2,4,6-trione (BA25). Isolated Yield 92%. ¹H-NMR (DMSO-d₆) δ 13.22 (1H, s), 10.81 (1H, s), 10.64 (1H, s), 9.65 (1H, s), 8.11 (1H, d, J=9.2 Hz), 6.82 (2H, d, J=9.2 Hz), and 2.60 (3H, s) ppm. ¹³C-NMR (DMSO-d₆) δ 175.9, 153.2, 150.4, 140.3, 126.7, 89.8, 16.3 ppm.

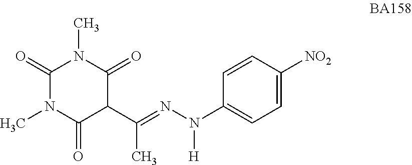 [SMILES: C/C(=N\Nc1ccc([N+](=O)[O-])cc1)C1C(=O)N(C)C(=O)N(C)C1=O]

1,3-Dimethyl-5-{(1E)-1-[2-(4-nitrophenyl)hydrazinylidene]ethyl}pyrimidine-2,4,6-trione (BA158). Isolated yield (92%). ¹H-NMR (DMSO-d₆) δ 13.15 (1H s), 9.95 (1H, s), 8, 12 (2H, d, J=9.2 Hz), 6.82 (2H, d, J=9.2 Hz), 3.12 (6H, s), and 2.55 (3H, s) ppm. ¹³C-NMR (DMSO-d₆) δ 176.1, 130.2, 131.3, 140.5, 126.3, 111.8, 89.2, 28.1, and 16.8 ppm.

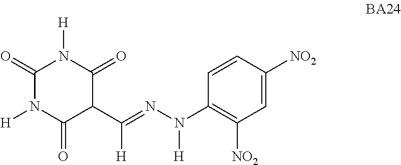 [SMILES: O=C1NC(=O)C(/C=N/Nc2ccc([N+](=O)[O-])cc2[N+](=O)[O-])C(=O)N1]

5-{(E)-[2-(2,4-Dinitrophenyl)hydrazinylidene]methyl}pyrimidine-2,4,6-trione (BA24). Isolated yield 96%. ¹H-NMR (DMSO-d₆) δ 10.90 (1H, s), 10.96 (2H, s), 8.84 (1H, d, J=2.8 Hz, 8.37 (1H, d of d, J₁=9.6 Hz, J2=2.4 Hz), 8.08 (1H, s), and 7.20 (1H, d, J=9.6 Hz) ppm. ¹³C-NMR (DMSO-d₆) δ 159.8, 151.5, 148.1, 138.3, 130.9. 130.6, 123.4, 116.8, and 92.00 ppm.

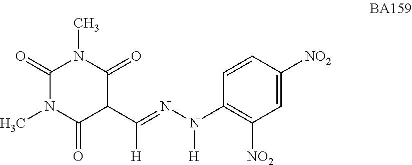 [SMILES: CN1C(=O)C(/C=N/Nc2ccc([N+](=O)[O-])cc2[N+](=O)[O-])C(=O)N(C)C1=O]

5-{(E)-[2-(2,4-dinitrophenyl)hydrazinylidene]methyl}-1,3-dimethylpyrimidine-2,4,6-trione (BA159). Isolated yield 97%. ¹H-NMR (DMSO-d₆) δ 11.51 (1H, s), 10.98 (1H, s), 8.37 (1H, d, J=9.2 Hz), 8.17 (1H, s), 7.20 (1H, d, J=9.2 Hz), and 3.11 (6H, s) ppm. ¹³C-NMR (DMSO-d₆) δ 160.4, 152.1, 147.8, 138.2, 130.8, 130.6, 123.3, 116.7, 115.6, 91.9, and 27.9 ppm.

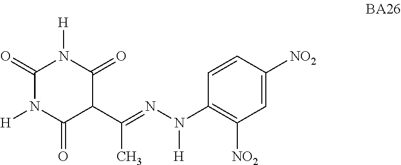 [SMILES: C/C(=N\Nc1ccc([N+](=O)[O-])cc1[N+](=O)[O-])C1C(=O)NC(=O)NC1=O]

5-{(1E)-1-[2-(2,4-Dinitrophenyl)hydrazinylidene]ethyl}pyrimidine-2,4,6-trione (BA26). Isolated yield 87%. ¹H-NMR (DMSO-d₆) δ 13.20 (1H, s), 10.93 (1H, s), 10.65 (1H, s), 10.57 (1H, s), 8.86 (1H, d, J=2.4 Hz), 8.35 (1H, d of d, J₁=9.6 Hz, J₂=2.4 Hz), 7.19 (1H, d, J=9.6 Hz) and 2.61 (3H, s) ppm. ¹³C-NMR (DMSO-d₆) δ 176.5, 150.3, 147.7, 138.4, 131.5, 131.1, 123.5, 116.1, 90.6, and 17.3 ppm.

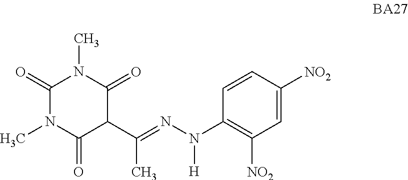 [SMILES: C/C(=N\Nc1ccc([N+](=O)[O-])cc1[N+](=O)[O-])C1C(=O)N(C)C(=O)N(C)C1=O]

5-{(1E)-1-[2-(2,4-dinitrophenyl)hydrazinylidene]ethyl}-1,3-dimethylpyrimidine-2,4,6-trione (BA27). Isolated yield 89%. ¹H-NMR (DMSO-d₆) δ 13.24 (1H, s), 10.64 (1H, s), 8.87 (1H, d, J=2.8 Hz), (1H, d of d, J₁=9.6 Hz, J₂=2.4 Hz), 7.16 (1H, d, J=9.6 Hz), 3.17 (6H, s), and 2.64 (3H, s) ppm. ¹³C-NMR (DMSO-d₆) δ 176.7, 151.3, 147.7, 138.5, 131.5, 131.1, 123.5, 116.1, 90.9, 28.3, and 17.4 ppm.

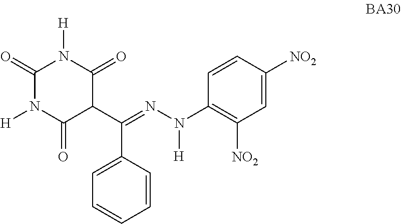 [SMILES: O=C1NC(=O)C(/C(=N/Nc2ccc([N+](=O)[O-])cc2[N+](=O)[O-])c2ccccc2)C(=O)N1]

5-[(Z)-[2-(2,4-Dinitrophenyl)hydrazinylidene](phenyl)methyl]pyrimidine-2,4,6-trione (BA30). Isolated yield 91%. ¹H-NMR (DMSO-d₆) δ 11.37 (1H s), 10.95 (2H, broad singlet), 8.87 (1H, d, J=2.8 Hz), 8.39 (1H, d of d, J₁=9.6 Hz, J₂=2.8 Hz) 8.12 (1H, d, J=9.6 Hz), 7.80 (2H, m), and 7.41 (3H, m). ¹³C-NMR (DMSO-d₆) δ 162.6, 151.4, 144.6, 137.7, 137.6, 130.8, 130.4, 129.9, 129.0, 128.1, 123.9, 117.3, and 82.7 ppm.

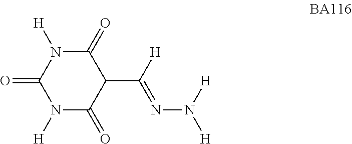 [SMILES: N/N=C/C1C(=O)NC(=O)NC1=O]

### Typical procedure for preparation of unsubstituted 2,4,6-pyrimidinetrione hydrazones

Preparation of 5-[(E)-hydrazinylidenemethyl]pyrimidine-2,4,6-trione (BA116). Methanol (200 ml) suspension of 5-formylpyrimidine-2,4,6-trione (1.56 g; 0.01 mol) and hydrazine (2 g; 0.04 mol) was refluxed for one hour sonicated for thirty minutes and refluxed for additional hour. Still hot yellow insoluble material was separated by filtration, washed with hot methanol (3×20 ml) and dried at 110° C. for ten minutes to give pure product (1.6 g; 94%). ¹H-NMR (DMSO-d₆) δ 11.01 (1H, broad s, 5-H), 10.55 (1H, s, NH), 10.49 (1H, s, NH), 7.97 (1H, s, CH), and 5.64 (2H, s, NH₂) ppm. ¹³C-NMR (DMSO-d₆) δ 161.9, 155.5, 151.6, and 87.5 ppm. Elemental Analysis: Calc: C, 35.30; H, 3.55; N, 32.93. Fund: C, 35.21; H, 3.61; N, 32.85.

5-[(E)-hydrazinylidenemethyl]-1,3-dimethylpyrimidine-2,4,6-trione (BA117). Yield (1.75 g; 88%). ¹H-NMR (DMSO-d₆) δ 11.01 (1H, broad s, 5-H), 8.05 (1H, s, CH), 5.69 (2H, s, NH₂), and 3.09 (6H, s, CH₃) ppm. ¹³C-NMR (DMSO-d₆) δ 168.9, 162.7, 161.6, 87.2, and 16.5 ppm.

5-[(1E)-1-hydrazinylideneethyl]pyrimidine-2,4,6-trione (BA118). Yield (1.78 g; 97%). ¹H-NMR (DMSO-d₆) δ 13.17 (1H, s, CH), 10.34 (2H, s, NH), 5.53 (2H, s, NH₂), and 2.59 (3H, s, CH₃) ppm. ¹³C-NMR (DMSO-d₆) δ 169.1, 166.3, 150.6, 86.7, and 15.8 ppm.

5-[(1E)-1-hydrazinylideneethyl]-1,3-dimethylpyrimidine-2,4,6-trione (BA119). Yield (1.9 g; 90%). ¹H-NMR (DMSO-d₆) δ 13.30 (1H, s, 5-H), 5.61 (2H, s, NH₂), 3.09 (6H, s NCH₃), and 2.61 (3H, s, CH₃) ppm. ¹³C-NMR (DMSO-d₆) δ 168.6, 164.0, 151.5, 87.1, 27.0, and 16.3 ppm.

5-[(E)-(2-methylhydrazinylidene)methyl]pyrimidine-2,4,6-trione (BA120). Yield (1.7 g; 93%). ¹H-NMR (DMSO-d₆) δ 10.92 (1H, s, 5-H), 10.61 (1H, s, NH), 10.56 (1H, s, NH), 8.01 (1H, s, CH), 5.82 (1H, q, J=5.6 Hz, NHN), and 2.58 (3H, d, J=4.8 Hz) ppm. ¹³C-NMR (DMSO-d₆) δ 166.1, 164.4, 156.6, 151.6, 88.3, and 39.7 ppm.

5-[(1E)-1-(2-methylhydrazinylidene)ethyl]pyrimidine-2,4,6-trione (BA121). Yield (1.8 g; 91%). ¹H-NMR (DMSO-d₆) δ 12.98 (1H, s, 5-H), 10.43 (2H, s, NH), 2.63 (3H, s, NCH₃), and 2.53 (3H, s, CH₃) ppm. ¹³C-NMR (DMSO-d₆) δ 162.9, 156.9, 152.2, 87.3, 39.6 and 17.7 ppm.

1,3-dimethyl-5-[(E)-(2-methylhydrazinylidene)methyl]pyrimidine-2,4,6-trione (BA122). Yield (1.95 g; 92%). ¹H-NMR (DMSO-d₆) δ 10.50 (1H, broad s, 5-H), 8.07 (1H, s, CH), 5.90 (1H, s, NH), 3.08 (6H, s, NCH₃), 2.61 (3H, s, NCH₃) ppm. ¹³C-NMR (DMSO-d₆) δ 163.0, 156.9, 152.2, 88.3, 39.6, and 27.6 ppm.

1,3-dimethyl-5-[(1E)-1-(2-methylhydrazinylidene)ethyl]pyrimidine-2,4,6(1H,3H,5H)-trione (BA123). Yield (2 g; 88%). 1H-NMR (CDCl3) 13.32 (1H, s, 5-H), 3.90 (1H, s, NH), 3.31 (6H, s, NCH3), 2.82 (3H, s, NCH3), and 2.77 (3H, s, CH3) ppm. 13C-NMR (CDCl3) 174.9, 163.0, 160.5, 39.2, 28.1, 27.8, and 16.7 ppm.

### Tables:

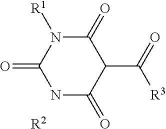

TABLE 1
5-Acyl-2,4,6-pyrimidinetriones

| | | | | MIC₈₀ | MIC₈₀ |
|---|---|---|---|---|---|
| Com- | | | | C. albicans | C. glabrata |
| pound | R¹ | R² | R³ | (μg/mL) | (μg/mL) |
| BA1 | H | H | C₆H₅ | — | — |
| BA2 | H | CH₃ | C₆H₅ | — | — |
| BA3 | CH₃ | CH₃ | C₆H₅ | — | — |
| BA4 | H | H | 3-O₂NC₆H₄ | — | — |
| BA5 | CH₃ | CH₃ | 3-O₂NC₆H₄ | — | — |
| BA6 | H | H | 4-O₂NC₆H₄ | — | — |
| BA7 | CH₃ | CH₃ | 4-O₂NC₆H₄ | — | — |
| BA8 | H | H | 3,5-(O₂N)₂C₆H₃ | — | — |
| BA9 | CH₃ | CH₃ | 3,5-(O₂N)₂C₆H₃ | — | — |
| BA10 | H | H | 4-HOC₆H₄ | — | — |
| BA11 | CH₃ | CH₃ | 4-HOC₆H₄ | — | — |
| BA12 | H | C₄H₉ | 4-HOC₆H₄ | — | — |
| BA13 | H | C₆H₅ | 4-HOC₆H₄ | — | — |
| BA14 | H | H | 4-CH₃OC₆H₄ | — | — |
| BA15 | CH₃ | CH₃ | 4-CH₃OC₆H₄ | — | — |
| BA16 | H | C₄H₉ | 4-CH₃OC₆H₄ | — | — |
| BA17 | CH₃ | CH₃ | 3-pyridinyl | — | — |
| BA18 | H | H | H | — | — |
| BA19 | CH₃ | CH₃ | H | — | — |
| BA20 | H | H | CH₃ | — | — |
| BA21 | H | C₆H₅ | CH₃ | — | — |

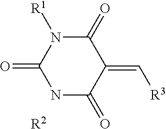

TABLE 2
5-Arylidene-2,4,6-pyrimidinetriones

| | | | | MIC₈₀ | MIC₈₀ |
|---|---|---|---|---|---|
| Com- | | | | C. albicans | C. glabrata |
| pound | R¹ | R² | R³ | (μg/mL) | (μg/mL) |
| BA39 | H | H | C₆H₅ | — | — |
| BA40 | H | H | 2-Naphthyl (C₁₀H₇) | 125 | — |
| BA41 | H | H | 1-Naphthyl (C₁₀H₇) | 125 | — |
| BA42 | H | H | CH═CH—C₆H₅ | 62 | |
| BA43 | CH₃ | CH₃ | CH═CH—C₆H₅ | 62 | 125 |
| BA44 | H | H | 4-HOC₆H₄ | — | — |
| BA45 | CH₃ | CH₃ | 4-HOC₆H₄ | — | — |
| BA46 | H | C₆H₅ | 4-HOC₆H₄ | — | — |
| BA47 | H | H | 2-HOC₆H₄ | — | 8 |
| BA48 | CH₃ | CH₃ | 2,4-(HO)₂C₆H₃ | — | 2 |
| BA50 | CH₃ | CH₃ | 4-CH₃OC₆H₄ | — | — |
| BA51 | CH₃ | CH₃ | 2,3,4-(CH₃O)₃C₆H₄ | — | — |
| BA52 | CH₃ | CH₃ | 2,4,6-(CH₃O)₃C₆H₄ | — | — |
| BA53 | H | H | 4-(CH₃)₂NC₆H₄ | 62 | — |
| BA54 | CH₃ | CH₃ | 4-(CH₃)₂NC₆H₄ | — | — |
| BA55 | H | H | 3-Furanyl (C₄H₃O) | — | — |
| BA56 | H | H | 2-Furanyl (C₄H₃O) | — | — |

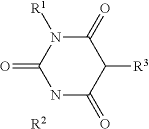

TABLE 3
5-Alkylated Pyrimidinetriones

| | | | | MIC₈₀ | MIC₈₀ |
|---|---|---|---|---|---|
| Com- | | | | C. albicans | C. glabrata |
| pound | R¹ | R² | R³ | (μg/mL) | (μg/mL) |
| BA60 | H | H | n-C₇H₁₅ | — | — |
| BA61 | H | C₆H₅ | CH(CH₃)₂ | — | — |
| BA62 | H | H | Cyclohexyl (C₆H₁₁) | — | — |
| BA63 | H | H | CH₂CH₂CH₂C₆H₁₁ | — | — |
| BA64 | H | H | CH₂C₆H₁₀-4-OH | — | — |
| BA65 | H | C₆H₅ | CH₂C₆H₁₀-4-OH | — | — |
| BA66 | H | H | CH₂CH₂CH₂C₆H₅ | — | — |
| BA67 | CH₃ | CH₃ | CH₂CH₂CH₂C₆H₅ | — | — |
| BA68 | H | H | CH₂C₆H₃-4-OH | — | — |
| BA69 | H | H | CH₂-2-C₁₀H₇ | — | — |

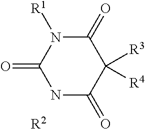

TABLE 4
5,5-Dialkylated Pyrimidinetriones

| | | | | | MIC₈₀ C. | MIC₈₀ C. |
|---|---|---|---|---|---|---|
| | | | | | albicans | glabrata |
| Compound | R¹ | R² | R³ | R⁴ | (μg/mL) | (μg/mL) |
| BA70 | H | H | CH₂C₆H₅ | CH₂C₆H₅ | — | — |
| BA71 | H | H | CH₂C₆H₄-4-N(CH₃)₂ | CH₂C₆H₄-4-N(CH₃)₂ | — | — |
| BA72 | H | H | CH₂CH₂CH₂C₆H₅ | CH₂C₆H₄-4-N(CH₃)₂ | — | — |

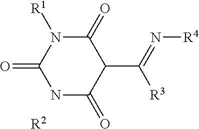

TABLE 5
Schiff Bases of 5-acylpyrimidinetriones

| | | | | | MIC₈₀ C. | MIC₈₀ C. |
|---|---|---|---|---|---|---|
| | | | | | albicans | glabrata |
| Compound | R¹ | R² | R³ | R⁴ | (μg/mL) | (μg/mL) |
| BA29 | H | H | CH₃ | (CH₂)₅CO₂H | — | — |
| BA35 | H | H | 4-CH₃OC₆H₄ | (CH₂)₅CO₂H | — | — |
| BA79 | CH₃ | CH₃ | H | 4-HOC₆H₄ | — | — |
| BA80 | CH₃ | CH₃ | CH₃ | 4-HOC₆H₄ | — | — |
| BA81 | H | H | H | 4-HOC₆H₄ | — | — |
| BA82 | CH₃ | CH₃ | H | 4-pyridinyl (C₅H₄N) | — | — |
| BA83 | H | H | H | 4-pyridinyl (C₅H₄N) | — | — |
| BA84 | CH₃ | CH₃ | CH₃ | 4-pyridinyl (C₅H₄N) | — | — |
| BA87 | H | H | CH₃ | 4-HOC₆H₄ | — | — |
| BA88 | H | H | CH₃ | 4-pyridinyl (C₅H₄N) | — | — |
| BA89 | H | H | H | C₆H₅ | — | — |
| BA90 | CH₃ | CH₃ | H | C₆H₅ | — | — |
| BA91 | CH₃ | CH₃ | CH₃ | C₆H₅ | — | — |
| BA92 | H | H | CH₃ | C₆H₅ | — | — |
| BA93 | H | H | H | 2-HOC₆H₄ | 8* | — |
| BA94 | CH₃ | CH₃ | H | 2-HOC₆H₄ | 8* | — |
| BA95 | CH₃ | CH₃ | H | 3-HOC₆H₄ | — | — |
| BA96 | H | H | H | 3-HOC₆H₄ | — | — |
| BA97 | H | H | H | 4-CH₃OC₆H₄ | — | |
| BA98 | CH₃ | CH₃ | H | 4-CH₃OC₆H₄ | — | |
| BA99 | CH₃ | CH₃ | CH₃ | 4-CH₃OC₆H₄ | — | |
| BA100 | H | H | CH₃ | 4-CH₃OC₆H₄ | — | |
| BA101 | H | H | H | 4-O₂NC₆H₄ | — | |
| BA102 | CH₃ | CH₃ | H | 4-O₂NC₆H₄ | — | |
| BA103 | H | H | H | 2-pyridinyl (C₅H₄N) | — | |
| BA104 | CH₃ | CH₃ | H | 2-pyridinyl (C₅H₄N) | — | |
| BA105 | H | H | H | 4-C₆H₄—C₆H₅ | — | |
| BA106 | CH₃ | CH₃ | H | 4-C₆H₄—C₆H₅ | — | |
| BA107 | H | H | H | 1,2,4-triazol-3-yl (C₂H₂N₃) | — | |
| BA108 | CH₃ | CH₃ | H | 1,2,4-triazol-3-yl (C₂H₂N₃) | — | |
| BA109 | H | H | H | 1,2,4-triazol-4-yl (C₂H₂N₃) | — | |
| BA110 | CH₃ | CH₃ | H | 1,2,4-triazol-4-yl (C₂H₂N₃) | — | |
| BA111 | H | H | H | 5-tetrazolyl (CN₄H) | — | |
| BA112 | CH₃ | CH₃ | H | 5-tetrazolyl (CN₄H) | — | |

*compound displayed MIC with <50% inhibition

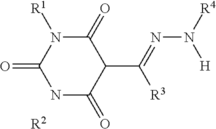

TABLE 6
Hydrazones of 5-Acylpyrimidinetriones

| | | | | | MIC₈₀ C. | MIC₈₀ C. |
|---|---|---|---|---|---|---|
| | | | | | albicans | glabrata |
| Compound | R¹ | R² | R³ | R⁴ | (μg/mL) | (μg/mL) |
| BA116 | H | H | H | H | 125 | |
| BA117 | CH₃ | CH₃ | H | H | 125 | |
| BA118 | H | H | CH₃ | H | — | |
| BA119 | CH₃ | CH₃ | CH₃ | H | — | |
| BA120 | H | H | H | CH₃ | — | |
| BA121 | H | H | CH₃ | CH₃ | — | |
| BA122 | CH₃ | CH₃ | H | CH₃ | 62* | |
| BA123 | CH₃ | CH₃ | CH₃ | CH₃ | — | |

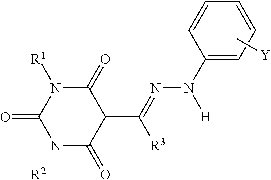

TABLE 7
Phenylhydrazones of 5-Acylpyrimidinetriones

| | | | | | MIC₈₀ C. | MIC₈₀ C. |
|---|---|---|---|---|---|---|
| | | | | | albicans | glabrata |
| Compound | R¹ | R¹ | R³ | Y | (μg/mL) | (μg/mL) |
| BA22 | H | H | H | 4-nitro (NO₂) | 2 | 1 |
| BA23 | CH₃ | CH₃ | H | 4-nitro (NO₂) | 2 | 2 |
| BA24 | H | H | H | 2,4-dinitro (NO₂)₂ | — | 125 |
| BA25 | H | H | CH₃ | 4-nitro (NO₂) | 2 | 2 |
| BA26 | H | H | CH₃ | 2,4-dinitro (NO₂)₂ | — | — |
| BA27 | CH₃ | CH₃ | CH₃ | 2,4-dinitro (NO₂)₂ | — | — |
| BA28 | H | C₆H₅ | CH₃ | 4-nitro (NO₂) | 31.5 | 16 |
| BA30 | H | H | C₆H₅ | 2,4-dinitro (NO₂)₂ | — | — |
| BA31 | H | C₄H₉ | C₆H₅ | 2,4-dinitro (NO₂)₂ | — | — |
| BA32 | H | C₆H₅ | C₆H₅ | 2,4-dinitro (NO₂)₂ | — | — |
| BA33 | CH₃ | CH₃ | C₆H₅ | 4-carboxy (COOH) | — | — |
| BA34 | CH₃ | CH₃ | 4-HOC₆H₄ | 2,4-dinitro (NO₂)₂ | — | — |
| BA38 | H | H | 5-pyrimidinetrione | 4-carboxy (COOH) | — | 125 |
| BA73 | CH₃ | CH₃ | H | H | 4 | 2 |
| BA74 | H | H | H | H | 4 | 2 |
| BA78 | CH₃ | CH₃ | CH₃ | H | 2 | 2 |
| BA85 | H | H | CH₃ | H | 2 | 2 |
| BA158 | CH₃ | CH₃ | CH₃ | 4-nitro (NO₂) | NT | |
| BA159 | CH₃ | CH₃ | H | 2,4-dinitro (NO₂)₂ | NT | |

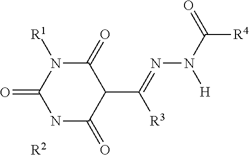

TABLE 8
N-Acylhydrazones of 5-Acylpyrimidinetriones

| | | | | | MIC₈₀ C. | MIC₈₀ C. |
|---|---|---|---|---|---|---|
| | | | | | albicans | glabrata |
| Compound | R¹ | R² | R³ | R⁴ | (μg/mL) | (μg/mL) |
| BA36 | CH₃ | CH₃ | 4-HOC₆H₄ | OCH₃ | — | — |
| BA37 | H | H | 4-O₂NC₆H₄ | OCH₃ | — | — |
| BA75 | CH₃ | CH₃ | CH₃ | OCH₃ | — | — |
| BA76 | CH₃ | CH₃ | H | OCH₃ | — | — |
| BA77 | H | H | H | OCH₃ | — | — |
| BA86 | H | H | CH₃ | OCH₃ | — | — |
| BA124 | H | H | H | C₆H₅ | — | |
| BA125 | CH₃ | CH₃ | H | C₆H₅ | — | |
| BA126 | H | H | CH₃ | C₆H₅ | — | |
| BA127 | CH₃ | CH₃ | CH₃ | C₆H₅ | — | |
| BA128 | H | H | H | 4-O₂NC₆H₄ | 62* | |
| BA129 | CH₃ | CH₃ | H | 4-O₂NC₆H₄ | 62* | |
| BA130 | H | H | CH₃ | 4-O₂NC₆H₄ | — | |
| BA131 | CH₃ | CH₃ | CH₃ | 4-O₂NC₆H₄ | — | |
| BA132 | H | H | H | 4-CH₃OC₆H₄ | — | |
| BA133 | CH₃ | CH₃ | H | 4-CH₃OC₆H₄ | 62* | |
| BA134 | H | H | CH₃ | 4-CH₃OC₆H₄ | — | |
| BA135 | CH₃ | CH₃ | CH₃ | 4-CH₃OC₆H₄ | — | |
| BA160 | H | H | H | (CH₂)₄CH₃ | NT | |
| BA161 | CH₃ | CH₃ | CH₃ | (CH₂)₄CH₃ | NT | |

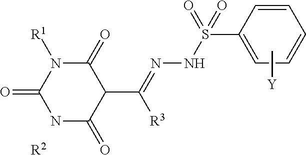

TABLE 9
Benzenesuifonohydrazones of 5-Acylpyrimidinetrione

| | | | | | MIC₈₀ C. | MIC₈₀ C. |
|---|---|---|---|---|---|---|
| | | | | | albicans | glabrata |
| Compound | R¹ | R² | R³ | Y | (μg/mL) | (μg/mL) |
| BA113 | H | H | H | 4-CH₃ | 16** | |
| BA114 | H | H | CH₃ | 4-CH₃ | — | |
| BA115 | CH₃ | CH₃ | H | 4-CH₃ | — | |
| BA136 | H | H | H | H | — | |
| BA137 | CH₃ | CH₃ | H | H | — | |
| BA138 | H | H | CH₃ | H | — | |
| BA139 | CH₃ | CH₃ | CH₃ | H | — | |

### REFERENCES

1. Eddouzi, J.; Parker, J. E.; Vale-Silva, L. A.; Coste, A.; Ischer, F.; Kelly, S.; Manai, M.; Sanglard, D. Molecular mechanisms of drug resistance in clinical Candida species isolated from Tunisian hospitals. Antimicrob. Agents Chemother. 2013, 57, 3182-3193.
2. Chakrabarti, A.; Chatterjee, S. S.; Shivaprakash, M. R. Overview of opportunistic fungal infections in India. Nippon Ishinkin. Gakkai Zasshi 2008, 49, 165-172.
3. Chandesris, M. O.; Lantemier, F.; Lecuit, M.; Lortholary, O. [Infectious complications of immune deficiencies]. Rev. Prat. 2007, 57, 1653-1664.
4. El-Cheikh, J.; Venton, G.; Crocchiolo, R.; Furst, S.; Faucher, C.; Granata, A.; Oudin, C.; Coso, D.; Bouabdallah, R.; Vey, N.; Duran, S.; Fougereau, E.; Berger, P.; Chabannon, C.; Blaise, D. Efficacy and safety of micafungin for prophylaxis of invasive fungal infections in patients undergoing haplo-identical hematopoietic SCT. Bone Marrow Transplant 2013.
5. Comely, O. A.; Vazquez, J.; De, W. J.; Betts, R.; Rotstein, C.; Nucci, M.; Pappas, P. G.; Ullmann, A. J. Efficacy of micafungin in invasive candidiasis caused by common Candida species with special emphasis on non-albicans Candida species. Mycoses 2013.
6. Comely, O. A.; Lasso, M.; Betts, R.; Klimko, N.; Vazquez, J.; Dobb, G.; Velez, J.; Williams-Diaz, A.; Lipka, J.; Taylor, A.; Sable, C.; Kartsonis, N. Caspofungin for the treatment of less common forms of invasive candidiasis. J Antimicrob. Chemother. 2007, 60, 363-369.
7. Xiang, M. J.; Liu, J. Y.; Ni, P. H.; Wang, S.; Shi, C.; Wei, B.; Ni, Y. X.; Ge, H. L. Erg11 mutations associated with azole resistance in clinical isolates of Candida albicans. FEMS Yeast Res 2013, 13, 386-393.
8. Bennett, J. E.; Izumikawa, K.; Man, K. A. Mechanism of increased fluconazole resistance in Candida glabrata during prophylaxis. Antimicrob. Agents Chemother. 2004, 48, 1773-1777.
9. Carradori, S.; Secci, D.; Bolasco, A.; Rivanera, D.; Mari, E.; Zicari, A.; Lotti, L. V.; Bizzarri, B. Synthesis and cytotoxicity of novel (thiazol-2-yl)hydrazine derivatives as promising anti-Candida agents. Eur. J Med. Chem 2013, 65, 102-111.
10. Altintop, M. D.; Ozdemir, A.; Turan-Zitouni, G.; Ilgin, S.; Atli, O.; Iscan, G.; Kaplancikli, Z. A. Synthesis and biological evaluation of some hydrazone derivatives as new anticandidal and anticancer agents. Eur. J Med. Chem 2012, 58, 299-307.
11. Padmaja, A.; Reddy, G. S.; Venkata Nagendra, M. A.; Padmavathi, V. Michael adducts-source for biologically potent heterocycles. Chem Pharm. Bull (Tokyo) 2008, 56, 647-653.
12. Padmavathi, V.; Reddy, G. D.; Venkatesh, B. C.; Padmaja, A. One-pot synthesis of 5-[[Bis(azolylmethylthio)]methylene]pyrimidine-2,4,6-(1H,3H,5H)-triones. Arch. Pharm. (Weinheim) 2011, 344, 165-169.
13. Dickey, J.; Gray, R. Barbituric Acid. Org Syn Coll 1943, 2, 60.
14. Hron, R. J.; Komati, R.; Jursic, B. Simple and efficient method for preparation of alkyl and aryl substituted ureas. Manuscript in preparation 2013.
15. Jursic, B.; Neumann, D. Preparation of 5-formyl- and 5-acetyl barbituric acids including the corresponding Schiff base and phenylhydrazones. Tetrahedron Lett 2001, 42, 8435-8439.
16. Wynberg, H. The Reimer-Tiemann Reaction. Chem Rev 1960, 60, 169-184.
17. Brown, D. The chemistry of heterocyclic compounds, the pyrimidines: Supplement 2; Wiley: New York, 1985; p 389.
18. Jursic B S; Neumann, D. M.; Bowdy, K. L.; Stevens, E. D. Simple, efficient, high yield syntheses of substituted and unsubstituted 5-benzoylbarbituric acids and their corresponding Schiff base. J Heterocyclic Chem 2004, 41, 233-246.
19. Jursic B S A simple method for Knoevenagel condensation of a,b-conjugated and aromatic aldehydes with barbituric acid. J Heterocyclic Chem 2001, 38, 655-657.
20. Jursic B S; Neumann, D. M. Preparation of 5,5′-pyrilidine and 5,5′-quinolidene bis-barbituric acid derivatives. J Heterocyclic Chem 2003, 40, 465-474.
21. Avonto, C.; Taglialatela-Scafati, O.; Pollastro, F.; Minassi, A.; Di, M., V; De, P. L.; Appendino, G. An NMR spectroscopic method to identify and classify thiol-trapping agents: revival of Michael acceptors for drug discovery? Angew. Chem Int. Ed Engl. 2011, 50, 467-471.
22. Deb, M.; Bhuyan, P. Uncatalyzed Knoevenagel condensation in aqueous media at room temperature. Tetrahedron Lett 2005, 46, 6453-6456.
23. Jursic, B.; Neumann, D. M. Reductive C-alkylation of barbituric acid derivatives with carbonyl compounds in the presence of platinum and palladium catalysts. Tetrahedron Lett 2001, 42, 4103-4107.
24. Jursic, B.; Neumann, D. Preparation of 5-formyl- and 5-acetyl barbituric acids including the corresponding Schiff base and phenylhydrazones. Tetrahedron Lett 2001, 42, 8435-8439.
25. Saha, A.; Kumar, R.; Kumar, R.; Devakumar, C. Development and assessment of green synthesis of hydrazides. Indian J Chem 2010, 49B, 526-531.
26. Verma, K.; Pandeya, S.; Singh, U.; Gupta, S.; Prashant, P.; Bhardwaj, G. Synthesis and pharmacological activity of some substituted menthone, semicarbazone and thiosemicarbazone derivatives. Int J Pharm Scien Nanotech 2009, 1, 357-362.
27. Pitucha, M.; Wujec, M.; Dobosz, M.; Kosikowska, U.; Malm, A. Synthesis and biological action of 1-aminomethyl derivatives of 3-R-4-phenyl-delta2-1,2,4-triazoline-5-thione. Acta Pol. Pharm 2005, 62, 443-449.
28. Hron, R. J.; Komati, R.; Jursic, B. Simple and efficient procedure for preparation of substituted carbazides. Manuscript in preparation 2013.
29. Clinical Laboratory and Standards Institute Reference method for Broth Dilution Antifungal Susceptibility Testing of Yeasts: Approved Standard. M27-A3 2010, 28.
30. Tzagoloff, A.; Dieckmann, C. L. PET genes of Saccharomyces cerevisiae. Microbiol. Rev 1990, 54, 211-225.
31. Hampsey, M. A review of phenotypes in Saccharomyces cerevisiae. Yeast 1997, 13, 1099-1133.
32. Gillum, A. M.; Tsay, E. Y.; Kirsch, D. R. Isolation of the Candida albicans gene for orotidine-5′-phosphate decarboxylase by complementation of S. cerevisiae ura3 and E. coli pyrF mutations. Mol. Gen. Genet. 1984, 198, 179-182.
33. Fidel, P. L., Jr.; Cutright, J. L.; Tait, L.; Sobel, J. D. A murine model of Candida glabrata vaginitis. J Infect. Dis. 1996, 173, 425-431.
34. Pegurier, C.; Collart, P.; Danhaive, P.; Defays, S.; Gillard, M.; Gilson, F.; Kogej, T.; Pasau, P.; Van, H. N.; Van, T. M.; van, K. B. Pyrazolone methylamino piperidine derivatives as novel CCR3 antagonists. Bioorg. Med. Chem Lett 2007, 17, 4228-4231.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

1. A composition, comprising an antimicrobial agent having the following structure: 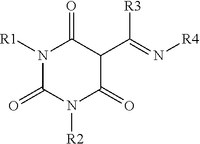
wherein R1 is selected from the group consisting of: H, alkyl, aryl, (CH₂)ₙCOOH with n=1-6, OH, and O-alkyl, where each of alkyl and aryl groups is independently optionally substituted or unsubstituted; wherein R2 is selected from the group consisting of: H, alkyl, aryl, (CH₂)ₙCOOH with n=1-6, OH, and O-alkyl, where each of alkyl and aryl groups is independently optionally substituted or unsubstituted; wherein R3 is selected from the group consisting of: H, alkyl, aryl, and COOH, where each of alkyl and aryl groups is independently optionally substituted or unsubstituted; wherein R4 is selected from the group consisting of: 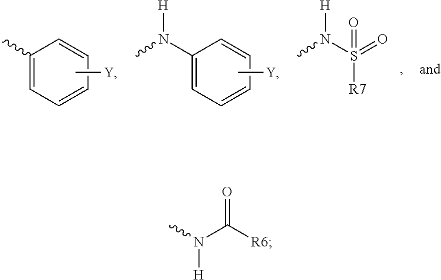
wherein Y is selected from the group consisting of: H, OH, NO₂, COOH, halogen, alkyl, and O-alkly, where each alkyl group is independently optionally substituted or unsubstituted, wherein R6 is selected from the group consisting of: alkyl, O-alkyl, O-aryl, and aryl, where each of alkyl and aryl groups is independently optionally substituted or unsubstituted; and wherein R7 is selected from the group consisting of: alkyl, O-alkyl, O-aryl, and aryl, where each of alkyl and aryl groups is independently optionally substituted or unsubstituted.

2. The composition of claim 1, wherein R1 is selected from the group consisting of H, methyl, and phenyl, wherein R2 is selected from the group consisting of: H, methyl, or phenyl, wherein R3 is selected from the group consisting of: H, methyl, phenyl group, 4-OHC₆H₄, 1-naphthyl, 2-naphthyl, CH═CH—C₆H₅, and 4-(CH₃)₂NC₆H₄.

3. The composition of claim 2, wherein if R4 is 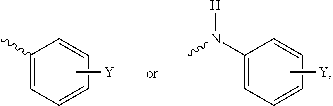
wherein the Y group is selected from the group consisting of 4-nitro, 4-methyl, and 4-carboxy, where the Y group is attached to the 4 position, wherein when two Y groups are present in the 2 and 4 position, the Y groups are selected from the group consisting of: 2,4-dinitro and 2,4-dichloro.

4. The composition of claim 2, wherein if R4 is 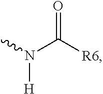
then R6 is selected from the group consisting of: O-methyl, methyl, O₂NC₆H₄, and CH₃OC₆H₄.

5. The composition of claim 2, wherein if R4 is 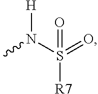
then R7 is selected from the group consisting of: methyl, phenyl, 4-O₂NC₆H₄, 4-CH₃C₆H₄, 4-CH₃OC₆H₄, 4-BrC₆H₄, and 1-naphthyl.

6. The composition of claim 2, wherein the antimicrobial agent has the following structure: 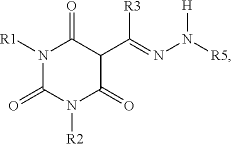
wherein R5 is selected from the group consisting of:

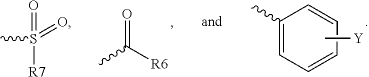

7. The composition of claim 2, wherein the antimicrobial agent has the following structure:

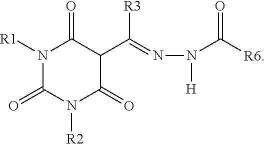

8. The composition of claim 2, wherein the antimicrobial agent has the following structure:

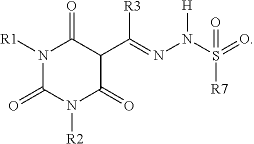

9. A pharmaceutical composition comprising a therapeutically effective amount of an antimicrobial agent, or a pharmaceutically acceptable salt of the antimicrobial agent, and a pharmaceutically acceptable carrier, to treat an infection, wherein the antimicrobial agent has the following structure: 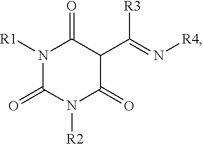
wherein R1 is selected from the group consisting of: H, alkyl, aryl, (CH₂)ₙCOOH with n=1-6, OH, and O-alkyl, where each of alkyl and aryl groups is independently optionally substituted or unsubstituted; wherein R2 is selected from the group consisting of: H, alkyl, aryl, (CH₂)ₙCOOH with n=1-6, OH, and O-alkyl, where each of alkyl and aryl groups is independently optionally substituted or unsubstituted; wherein R3 is selected from the group consisting of: H, alkyl, aryl, and COOH, where each of alkyl and aryl groups is independently optionally substituted or unsubstituted; wherein R4 is selected from the group consisting of: 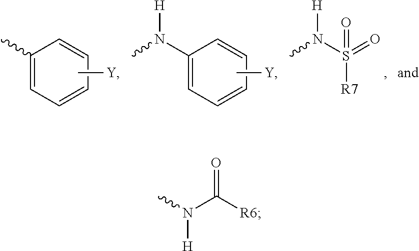
wherein Y is selected from the group consisting of: H, OH, NO₂, COOH, halogen, alkyl, and O-alkly, where each alkyl group is independently optionally substituted or unsubstituted, wherein R6 is selected from the group consisting of: alkyl, O-alkyl, O-aryl, and aryl, where each of alkyl and aryl group is independently optionally substituted or unsubstituted; and wherein R7 is selected from the group consisting of: alkyl, O-alkyl, O-aryl, and aryl, where each of alkyl and aryl groups is independently optionally substituted or unsubstituted.

10. The pharmaceutical composition of claim 9, wherein the infection is selected from the group selected from: a fungal infection, a bacterial infection, and a combination thereof.

11. The pharmaceutical composition of claim 9, wherein the infection is a fungal infection.

12. The pharmaceutical composition of claim 9, wherein the infection caused by an azole-resistant fungus.

13. The pharmaceutical composition of claim 11, wherein R1 is selected from the group consisting of: H, methyl, and phenyl, wherein R2 is selected from the group consisting of: H, methyl, or phenyl, wherein R3 is selected from the group consisting of: H, methyl, phenyl group, 4-OHC₆H₄, 1-naphthyl, 2-naphthyl, CH═CH—C₆H₅, and 4-(CH₃)₂NC₆H₄.

14. The pharmaceutical composition of claim 11, wherein if R4 is 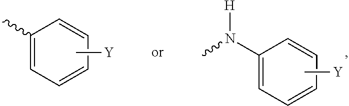
then the Y group is selected from the group consisting of 4-nitro, 4-methyl, and 4-carboxy, where the Y group is attached to the 4 position, wherein when two Y groups are present in 2 and 4 positions, the Y groups are selected from the group consisting of: 2,4-dinitro and 2,4-dichloro.

15. The pharmaceutical composition of claim 11, wherein if R4 is 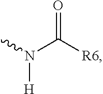
then R6 is selected from the group consisting of: O-methyl, methyl, O₂NC₆H₄, and CH₃OC₆H₄.

16. The pharmaceutical composition of claim 11, wherein if R4 is 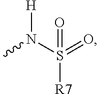
then R7 is selected from the group consisting of: methyl, phenyl, 4-O₂NC₆H₄, 4-CH₃C₆H₄, 4-CH₃OC₆H₄, 4-BrC₆H₄, and 1-naphthyl.

17. A method of treating an infection comprising: delivering to a subject in need thereof, a pharmaceutical composition, wherein the pharmaceutical composition includes a therapeutically effective amount of an antimicrobial agent, or a pharmaceutically acceptable salt of the antimicrobial agent, and a pharmaceutically acceptable carrier, to treat the infection, wherein the antimicrobial agent has the following structure: 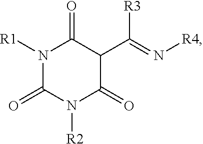
wherein R1 is selected from the group consisting of: H, alkyl, aryl, (CH₂)ₙCOOH with n=1-6, OH, and O-alkyl, where each of alkyl and aryl groups is independently optionally substituted or unsubstituted; wherein R2 is selected from the group consisting of: H, alkyl, aryl, (CH₂)ₙCOOH with n=1-6, OH, and O-alkyl, where each of alkyl and aryl groups is independently optionally substituted or unsubstituted; wherein R3 is selected from the group consisting of: H, alkyl, aryl, and COOH, where each of alkyl and aryl groups is independently optionally substituted or unsubstituted; wherein R4 is selected from the group consisting of: 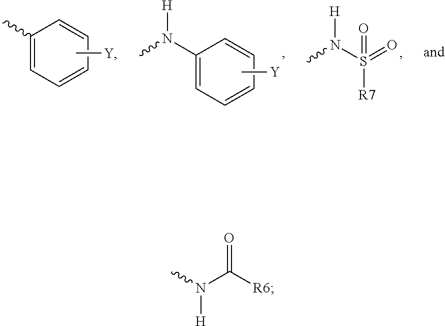
wherein Y is selected from the group consisting of: H, OH, NO₂, COOH, halogen, alkyl, and O-alkly, where each alkyl group is independently optionally substituted or unsubstituted, wherein R6 is selected from the group consisting of: alkyl, O-alkyl, O-aryl, and aryl, where each of alkyl and aryl groups is independently optionally substituted or unsubstituted; and wherein R7 is selected from the group consisting of: alkyl, O-alkyl, O-aryl, and aryl, where each of alkyl and aryl groups is independently optionally substituted or unsubstituted.

18. The method of claim 17, wherein the infection is selected from the group selected from: a fungal infection, a bacterial infection, and a combination thereof.

19. The method of claim 17, wherein the infection is a fungal infection.

20. The method of claim 17, wherein the infection caused by an azole-resistant fungus.

21. The method of claim 17, wherein the infection is a Candida spp. fungal infection.

22. The method of claim 19, wherein R1 is selected from the group consisting of: H, methyl, and phenyl, wherein R2 is selected from the group consisting of: H, methyl, or phenyl, wherein R3 is selected from the group consisting of: H, methyl, phenyl group, 4-OHC₆H₄, 1-naphthyl, 2-naphthyl, CH═CH—C₆H₅, and 4-(CH₃)₂NC₆H₄.

23. The method of claim 22, wherein if R4 is 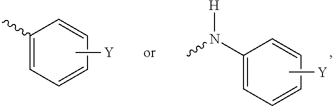
then the Y group is selected from the group consisting of 4-nitro, 4-methyl, and 4-carboxy, where the Y group is attached to the 4 position, wherein when two Y groups are present in 2 and 4 position, the Y groups are selected from the group consisting of: 2,4-dinitro and 2,4-dichloro.

24. The method of claim 22, wherein if R4 is 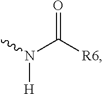
then R6 is selected from the group consisting of: O-methyl, methyl, O₂NC₆H₄, and CH₃OC₆H₄.

25. The method of claim 22, wherein if R4 is 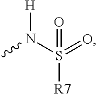
then R7 is selected from the group consisting of: methyl, phenyl, 4-O₂NC₆H₄, 4-CH₃C₆H₄, 4-CH₃OC₆H₄, 4-BrC₆H₄, and 1-naphthyl.
